SEQUENCE LISTING (27 sequences)

SEQ ID NO: 1 - DNA, 14 bp, Artificial Sequence
  adaptor common strand
gactggcagctcgt

SEQ ID NO: 2 - DNA, 28 bp, Artificial Sequence
  encoded adaptor
nnnaacgagctgccagtccatttaggcg

SEQ ID NO: 3 - DNA, 28 bp, Artificial Sequence
  encoded adaptor
nnngacgagctgccagtcctgattaccg

SEQ ID NO: 4 - DNA, 28 bp, Artificial Sequence
  encoded adaptor
nnncacgagctgccagtcaccaatacgg

SEQ ID NO: 5 - DNA, 28 bp, Artificial Sequence
  encoded adaptor
nnntacgagctgccagtccgctttgtag

SEQ ID NO: 6 - DNA, 28 bp, Artificial Sequence
  encoded adaptor
nnanacgagctgccagtcggaacctgaa

SEQ ID NO: 7 - DNA, 28 bp, Artificial Sequence
  encoded adaptor
nngnacgagctgccagtctgtgcgtgat

SEQ ID NO: 8 - DNA, 28 bp, Artificial Sequence
  encoded adaptor
nncnacgagctgccagtcaccgacattc

SEQ ID NO: 9 - DNA, 28 bp, Artificial Sequence
  encoded adaptor
nntnacgagctgccagtcattcctcctc

SEQ ID NO: 10 - DNA, 28 bp, Artificial Sequence
  encoded adaptor
nannacgagctgccagtccgaagaagtc

SEQ ID NO: 11 - DNA, 28 bp, Artificial Sequence
  encoded adaptor
ngnnacgagctgccagtctggtctctct

SEQ ID NO: 12 - DNA, 28 bp, Artificial Sequence
  encoded adaptor
ncnnacgagctgccagtctagcggactt

SEQ ID NO: 13 - DNA, 28 bp, Artificial Sequence
  encoded adaptor
ntnnacgagctgccagtcggcgataact

SEQ ID NO: 14 - DNA, 28 bp, Artificial Sequence
  encoded adaptor
annnacgagctgccagtcgcatccatct

SEQ ID NO: 15 - DNA, 28 bp, Artificial Sequence
  encoded adaptor
gnnnacgagctgccagtccaactcgtca

SEQ ID NO: 16 - DNA, 28 bp, Artificial Sequence
  encoded adaptor
cnnnacgagctgccagtccacagcaaca

SEQ ID NO: 17 - DNA, 28 bp, Artificial Sequence
  encoded adaptor
tnnnacgagctgccagtcgccagtgtta

SEQ ID NO: 18 - DNA, 14 bp, Artificial Sequence
  adaptor
gactggcagctcgt

SEQ ID NO: 19 - DNA, 17 bp, Artificial Sequence
  adaptor
atcacgagctgccagtc

SEQ ID NO: 20 - DNA, 16 bp, Artificial Sequence
  adaptor
gactggcagcagtcgt

SEQ ID NO: 21 - DNA, 19 bp, Artificial Sequence
  adaptor
atcacgactgctgccagtc

SEQ ID NO: 22 - DNA, 18 bp, Artificial Sequence
  adaptor
dgggaaaaaaaaaaaaaa

SEQ ID NO: 23 - DNA, 10 bp, Artificial Sequence
  adaptor
tttttttttt

SEQ ID NO: 24 - DNA, 24 bp, Artificial Sequence
  carrier DNA
agtgaacctcgttagccagcaatc

SEQ ID NO: 25 - DNA, 10 bp, Artificial Sequence
  template DNA
agtcctaagg

SEQ ID NO: 26 - DNA, 18 bp, Artificial Sequence
  adaptor
nnnnacgagctgccagtc

SEQ ID NO: 27 - DNA, 28 bp, Artificial Sequence
  synthetic conjugate
agtcctaagggtaaacgagctgccagtc

(19) United States
(12) Patent Application Publication
(10) Pub. No.: US 2003/0224419 A1
(43) Pub. Date: Dec. 4, 2003

(54) Data analysis and display system for ligation-based DNA sequencing

(75) Inventors: Kevin C. Corcoran, Hayward, CA (US); Sam Eletr, Kensington, CA (US)

Correspondence Address: PERKINS COIE LLP, P.O. BOX 2168, MENLO PARK, CA, 94026, US

(73) Assignee: Lynx Therapeutics, Inc.

(21) Appl. No.: 10/407,089

(22) Filed: Apr. 2, 2003

Related U.S. Application Data
(63) Continuation of application No. 09/654,187, filed on Sep. 1, 2000, now abandoned.
Application No. 09/654,187 is a division of application No. 08/862,610, filed on May 23, 1997, now abandoned.
(60) Provisional application No. 60/182,454, filed on Feb. 15, 2000.

(30) Foreign Application Priority Data
May 22, 1998 (WO) PCT/US98/11224

(51) Int. Cl. C12Q 1/68; G06F 19/00; G01N 33/48; G01N 33/50

(52) U.S. Cl. 435/6; 702/20

(57) ABSTRACT

A data analysis system for ligation-based sequencing is provided. The system includes a base calling algorithm that may be implemented with a program of instructions (e.g., software) for determining a signature of a nucleotide sequence. A graphical user interface (GUI) enables a user to interact with and control various aspects of the base calling algorithm. The base calling algorithm and associated software may be used in connection with a technique that combines non-gel-based signature sequencing with in vitro cloning of millions of templates on separate 5 μm diameter microbeads.

### CROSS REFERENCE TO RELATED APPLICATIONS

This application claims priority on: (1) U.S. provisional patent application serial No. 60/182,454, filed Feb. 15, 2000; (2) international patent application PCT/US98/11224, filed May 22, 1998; and (3) U.S. utility patent application Ser. No. 08/862,610, filed May 23, 1997. The contents of each of these priority applications is incorporated herein by reference.

### FIELD OF THE INVENTION

The invention relates to a system, method and apparatus for carrying out massively parallel signature sequencing (MPSS) analysis on microbead arrays. More particularly, the invention relates to a base calling and signature sequencing technique, which may be implemented with a program of instructions and graphical user interface (GUI) running on a computer system.

Documents

[1] Lander, E. S. The new genomics: global views of biology. Science 274: 536-539 (1996).

[2] Collins, F. S., et al. New goals for the U.S. human genome project: 1998-2003 (1998). Science 282: 682-689.

[3] Duggan, D. J., Bittner, M., Chen, Y., Meltzer, P. & Trent, J. M. Expression profiling using cDNA microarrays. Nature Genet. 21: 10-14 (1999).

[4] Hacia, J. G. Resequencing and mutational analysis using oligonucleotide microarrays. Nature Genet. 21: 42-47 (1999).

[5] Okubo, K. et al. Large scale cDNA sequencing for analysis of quantitative and qualitative aspects of gene expression. Nature Genet. 2: 173-179 (1992).

[6] Velculescu, V. E., Zhang, L., Vogelstein, B. & Kinzler, K. W. Serial analysis of gene expression. Science 270: 484-487 (1995).

[7] Bachem, C. W. B. et al. Visualization of differential gene expression using a novel method of RNA fingerprinting based on AFLP: analysis of gene expression during potato tuber development. Plant J. 9: 745-753 (1996).

[8] Shimkets, R. A. et al. Gene expression analysis by transcript profiling coupled to gene database query. Nat. Biotechnol. 17: 798-803 (1999).

[9] Audic, S. & Claverie, J. The significance of digital gene expression profiles. Genome Res. 7: 986-995 (1997).

[10] Wittes, J. & Friedman, H. P. Searching for evidence of altered gene expression: a comment on statistical analysis of microarray data. J. Natl. Cancer Inst. 91: 400-401 (1999).

[11] Richmond, C. S., Glasner, J. D., Mau, R., Jin, H. & Blattner, F. R. Genome-wide expression profiling in Escherichia coli K-12. Nucleic Acids Res. 27: 3821-3835 (1999).

[12] Brenner, S. et al. In vitro cloning of complex mixtures of DNA on microbeads: physical separation of differentially expressed cDNAs. Proc. Natl. Acad. Sci. USA 97: 1655-1670 (2000).

[13] Velculescu, V. E. et al. 1997. Characterization of the yeast transcriptome. Cell 88: 243-251 (1997).

[14] Feller, W. An Introduction to Probability Theory and Its Applications, Vol. I, Third Edition (John Wiley & Sons, Inc., New York, 1968).

[15] Altschul, S. F. et al. Gapped BLAST and PSI-BLAST: a new generation of protein database search programs. Nucleic Acids Res. 25: 3389-3402 (1997).

[16] Chervitz, S. A. et al. Using the Saccharomyces Genome Database (SGD) for analysis of protein similarities and structure. Nucleic Acids Res. 27: 74-78 (1999).

[17] Brewster, N. K., Val, D. L., Walker, M. E. & Wallace, J. C. Regulation of pyruvate carboxylase isozyme (PYC1, PYC2) gene expression in Saccharomyces cerevisiae during fermentative and nonfermentative growth. Archives Biochem. Biophys. 311: 62-71 (1994).

### BACKGROUND OF THE INVENTION

After the first complete sequence of a human genome is obtained, the next challenge will be to discover and understand the function and variation of genes and, ultimately, to understand how such qualities affect health and disease. [1, 2] A key to this undertaking will be the availability of methods for efficient and accurate identification of genetic variation and expression patterns among large sets of genes. [2] Several powerful techniques have been developed for such analyses that depend either on specific hybridization of probes to microarrays [3, 4] or on the counting of tags or signatures of DNA fragments. [5-8] While the former provides the advantages of scale and the capability of detecting a wide range of gene expression levels, such measurements are subject to variability relating to probe hybridization differences and cross-reactivity, element-to-element differences within microarrays, and microarray-to-microarray differences. [9-11] On the other hand, the latter methods, which provide digital representations of abundance, are statistically more robust; they do not require repetition or standardization of counting experiments, as counting statistics are well-modeled by the Poisson distribution, and the precision and accuracy of relative abundance measurements may be increased by increasing the size of the sample of tags or signatures counted. [9] Unfortunately, however, this property is difficult to realize routinely because of the cost and scale of effort required.

Some of these difficulties have been addressed by the development of a new sequencing approach referred to as massively parallel signature sequencing, or MPSS, which uses a novel ligation-based sequencing scheme to identify simultaneously signatures of very large numbers of DNAs attached to microbeads disposed in a closely packed array. A challenge to this new sequencing approach has been the contribution to noise that repeated use of ligation and cleavage enzymes makes to measurements indicating the presence of a particular nucleotide at a particular location.

### SUMMARY OF THE INVENTION

Accordingly, one of the objects of the present invention is to provide a system, method and apparatus for determining a signature of a nucleotide sequence using a base calling algorithm in a ligation-based sequencing method.

It is another object of this invention to provide a program of instructions and a graphical user interface (GUI) (e.g., software) for implementing, and enabling user interaction with, such a base calling algorithm.

In one aspect, the invention includes a method of determining a nucleotide sequence of a polynucleotide from a series of optical measurements. Such series of measurements comprise a plurality of groups wherein each group contains one or more sets of four optical measurements and each optical measurement within a set corresponds to a different one of deoxyadenosine, deoxyguanosine, deoxycytidine, or deoxythymidine. The groups of optical measurements are produced by successively ligating to and cleaving from the end of a target polynucleotide signal-generating adaptor having protruding stands, such as the encoded adaptors described more fully below. Preferably, each optical measurement has a value, such as fluorescence intensity, and each set of optical measurements corresponds to a separate nucleotide position of the protruding strand of the signal-generating adaptor. Preferably, the method is implemented by the steps of (i) adjusting the value of the optical measurements of each set within a group by repeatedly subtracting therefrom a predetermined fraction of the value of the corresponding optical measurement of the corresponding set obtained in the previous ligation until the ratio of the highest value to the next highest value in the same set is greater than or equal to a first predetermined fraction, or until the sum of the repeatedly subtracted fractions is less than or equal to a predetermined factor; and (ii) assigning a base code to each set based on the results of the adjusting. Preferably, the plurality of groups is 3, 4, or 5, and the number of nucleotide positions in the protruding strand of the signal-generating adaptor is from 1 to 5.

In another aspect, the invention involves a method for determining a signature of a nucleotide sequence. The method comprises obtaining optical measurements having values ^{j}vᵢ₁, ^{j}vᵢ₂, ^{j}vᵢ₃, and ^{j}vᵢ₄ indicative of each nucleotide in each of a j^{th} group of nucleotide positions i, for i equal 1 through k and for j equal 1 through m; for every group of nucleotide positions from j equal 2 through m, and every position from i equal 1 through k, adjusting the values ^{j}vᵢ₁, ^{j}vᵢ₂, ^{j}vᵢ₃, and ^{j}vᵢ₄ by repeatedly subtracting from each a first predetermined fraction of ^{j-1}vᵢ₁, ^{j-1}vᵢ₂, ^{j-1}vᵢ₃, and ^{j-1}vᵢ₄, respectively, until the ratio of the highest value in the set of ^{j}vᵢ₁ through ^{j}vᵢ₄, to the next highest value in the same set is greater than or equal to a predetermined factor, or until the repeatedly subtracted fractions have a sum equal to a second predetermined fraction; and generating a base call for position i in the j^{th} group based on results of the adjusting.

Preferably, the base call generating comprises assigning a base code corresponding to the highest value to position i in the j^{th} group whenever the highest value is greater than or equal to a predetermined minimum value and the ratio of the highest value in the set of ^{j}vᵢ₁ through ^{j}vᵢ₄, to the next highest value in the same set is greater than or equal to the predetermined factor, and assigning a two-base ambiguity code corresponding to the highest value and the next highest value whenever the ratio is less than the predetermined factor and the highest value and the next highest value are each greater than or equal to the predetermined minimum value.

The method may further comprise rejecting the signature whenever the number of ambiguity codes assigned is greater than one.

In a preferred embodiment, the obtaining of optical measurements comprises adjusting values ^{j}vᵢ₁, ^{j}vᵢ₂, ^{j}vᵢ₃, and ^{j}vᵢ₄, for i equal 1 through k and for j equal 1 through m, for background noise, which is computed as the average of the lowest three of ^{j}vᵢ₁, ^{j}vᵢ₂, ^{j}vᵢ₃, and ^{j}vᵢ₄, and subtracted from each of ^{j}vᵢ₁, ^{j}vᵢ₂, ^{j}vᵢ₃, and ^{j}vᵢ₄.

The nucleotide groups, j=1 through m, are preferably contiguous, with m=3, 4 or 5, and the number of nucleotides in a group k=1, 2, 3, 4 or 5.

Preferably, the predetermined factor is between about 2 and about 5, the predetermined minimum value is greater than 125% of the background noise, the first predetermined fraction is 1/50, and the second predetermined fraction is set such that the highest value does not fall below 125% of the background noise.

According to another aspect of the invention, an apparatus for determining a signature of a nucleotide sequence is provided. The apparatus comprises a storage medium that stores a plurality of sets of digital signal values ^{j}vᵢ₁, ^{j}vᵢ₂, ^{j}vᵢ₃, and ^{j}vᵢ₄ indicative of each nucleotide in each of a j^{th} group of nucleotide positions i, for i=1 through k and for j equal 1 through m; and a processor in communication with the storage medium. The processor is operable to adjust the values ^{j}vᵢ₁, ^{j}vᵢ₂, ^{j}vᵢ₃, and ^{j}vᵢ₄, for every nucleotide position from i equal 1 through k in every group of nucleotide positions from j equal 2 through m, by repeatedly subtracting from each a first predetermined fraction of ^{j-1}vᵢ₁, ^{j-1}vᵢ₂, ^{j-1}vᵢ₃, and ^{j-1}vᵢ₄, respectively, until the ratio of the highest value in the set of ^{j}vᵢ₁ through ^{j}vᵢ₄, to the next highest value in the same set is greater than or equal to a predetermined factor, or until the repeatedly subtracted fractions have a sum equal to a second predetermined fraction, and generate a base call for position i in the j^{th} group based on results of the adjusting.

To generate a base call for position i in the j^{th} group, the processor preferably assigns a base code corresponding to the highest value to position i in the j^{th} group whenever the highest value is greater than or equal to a predetermined minimum value and the ratio of the highest value in the set of ^{j}vᵢ₁ through ^{j}vᵢ₄, to the next highest value in the same set is greater than or equal to the predetermined factor, and assigns a two-base ambiguity code corresponding to the highest value and the next highest value whenever the ratio is less than the predetermined factor and the highest value and the next highest value are each greater than or equal to the predetermined minimum value.

In a preferred embodiment, the processor renders a graphical representation of the digital signal values on the display upon user command, and renders a graphical representation of a plurality of microbeads, each containing at least one copy of the nucleotide sequence, on the display upon user command.

According to another aspect of the invention, a system for determining a signature of a nucleotide sequence is provided. The system comprises a processing and detection apparatus including an optical train operable to collect and convert a plurality of optical signals into corresponding digital signal values that comprise a plurality of sets digital signal values ^{j}vᵢ₁, ^{j}vᵢ₂, ^{j}vᵢ₃, and ^{j}vᵢ₄ indicative of each nucleotide in each of a j^{th} group of nucleotide positions i, for i=1 through k and for j equal 1 through m; a storage medium that stores ^{j}vᵢ₁, ^{j}vᵢ₂, ^{j}vᵢ₃, and ^{j}vᵢ₄; and a processor, operable as described above, in communication with the storage medium.

The processor's functions may be specified by a program of instructions that are executed by the processor. The program of instructions may be embodied in software, or in hardware formed integrally or in communication with the processor.

Preferably, the system further comprises a display and a graphical user interface presented on the display for enabling a user to display and manipulate data and results.

A data base, in communication with the processor, may be used for storing sequencing information, and a second processor in communication with the data base used for performing quality control analysis on the sequence signature.

In yet another aspect, the invention involves a processor-readable medium embodying a program of instructions for execution by a processor for performing the above-described method of determining a signature of a nucleotide sequence.

Still another aspect of the invention involves a graphical user interface presented on a computer for facilitating interaction between a user and a computer-implemented method of determining a signature of a nucleotide sequence. In one embodiment, the graphical user interface comprises a data display area for displaying one or more displays of data; and a control area for displaying selectable functions including a first function which when selected causes a graphical representation of the plurality of digital signal values to be displayed in the data display area, and a second function which when selected causes a graphical representation of a plurality of sequence-containing microbeads to be displayed in the data display area.

The selectable functions may be represented by graphical push buttons displayed in the control area of the graphical user interface.

In another embodiment, the graphical user interface comprises an animation mode including a first main window having a display area for displaying an animated image of a sequence-containing bead array, and a first control panel for displaying one or more selectable functions associated with the animation mode; an alignment mode including a second main window for aligning shifted images to show bead movement based on a comparison with a reference image, and a second control panel for displaying one or more selectable functions associated with the alignment mode; and a bead mode including a third main window for displaying a sequence-containing bead array, and one or more selectable functions for performing one or more base calling functions.

### BRIEF DESCRIPTION OF THE FIGURES

FIG. 1 is a flow chart illustrating the general signature sequencing process, according to embodiments of the invention.

FIG. 2 is a schematic illustration of various components of a system that may be used to carry out the signature sequencing operations, according to embodiments of the invention.

FIG. 3 is a block diagram of various components in a computer system that may be used to carry out various aspects of the invention.

FIG. 4 is a schematic illustration of sequence determination using the type IIs restriction endonuclease BbvI.

FIG. 5 is a schematic illustration of the process of using encoded adaptors to identify four bases in each ligation-cleavage cycle.

FIG. 6A is a longitudinal cross-sectional view of a flow chamber or cell, constructed in accordance with the invention and showing microparticles being loaded into the cell.

FIG. 6B is a top view of the flow cell.

FIG. 6C is a lateral cross-sectional view of the flow cell.

FIG. 7 is a schematic and functional representation of a system, including the flow cell, as well as detection, imaging and analysis components, for carrying out various aspects of the present invention.

FIGS. 8 and 9 depict a diagram of a false-color microbead array with an insert showing raw signature data from the microbead at the indicated position, with the called base shown above each histogram set.

FIG. 10 is a flow chart illustrating a sequencing method, according to embodiments of the invention.

FIG. 11 is a flow chart illustrating the signal processing and base calling aspects of the signature sequencing method, according to embodiments of the invention.

FIGS. 12A through 12T illustrate various aspects of a graphical user interface (GUI) for the base calling algorithm, according to embodiments of the invention.

### DETAILED DESCRIPTION OF THE INVENTION

I. Definitions

The term “oligonucleotide” as used herein includes linear oligomers of natural or modified monomers or linkages, including deoxyribonucleosides, ribonucleosides, anomeric forms thereof, peptide nucleic acids (PNAs), and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units, e.g. 40-60. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. Usually oligonucleotides of the invention comprise the four natural nucleotides; however, they may also comprise non-natural nucleotide analogs. It is clear to those skilled in the art when oligonucleotides having natural or non-natural nucleotides may be employed, e.g. where processing by enzymes is called for, usually oligonucleotides consisting of natural nucleotides are required.

The term “oligonucleotide tag(s)” as used herein refers to an oligonucleotide to which a oligonucleotide tag specifically hybridizes to form a perfectly matched duplex or triplex. Where specific hybridization results in a triplex, the oligonucleotide tag may be selected to be either double-stranded or single-stranded. Thus, where triplexes are formed, the term “complement” is meant to encompass either a double-stranded complement of a single-stranded oligonucleotide tag or a single-stranded complement of a double-stranded oligonucleotide tag.

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex. Conversely, a “mismatch” in a duplex between a tag and an oligonucleotide means that a pair or triplet of nucleotides in the duplex or triplex fails to undergo Watson-Crick and/or Hoogsteen and/or reverse Hoogsteen bonding.

As used herein, “nucleoside” includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the only proviso that they are capable of specific hybridization. Such analogs include synthetic nucleosides designed to enhance binding properties, reduce complexity, increase specificity, and the like.

II. System Overview

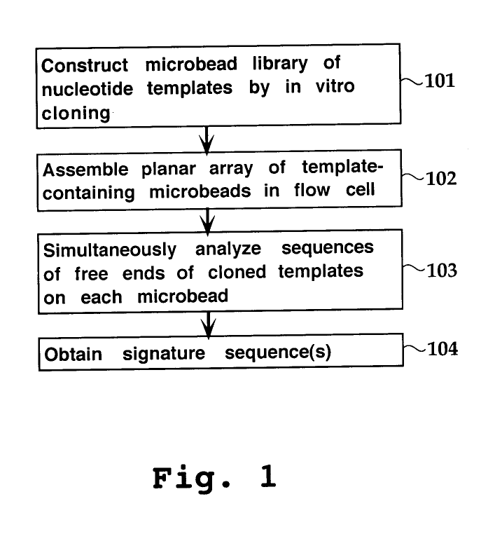

The present invention provides a base calling algorithm for a ligation-based sequencing method, and a program of instructions including a GUI for implementing and controlling the base calling algorithm. Preferably, the invention is employed with the DNA sequencing process illustrated in FIG. 1.

The flow chart of FIG. 1 illustrates the general signature sequencing process. The process begins in step 101 by constructing a microbead library of nucleotide (e.g. DNA) templates. Next, in step 102, a planar array of template-containing microbeads is assembled in a flow cell. Sequences of the free ends of the cloned templates on each microbead are then simultaneously analyzed in step 103 using a fluorescence-based, ligation-based sequencing method that does not require DNA fragment separation to obtain sequence information (step 104). In accordance with the invention, the sequencing method includes the base calling algorithm and associated GUI.

III. System Components

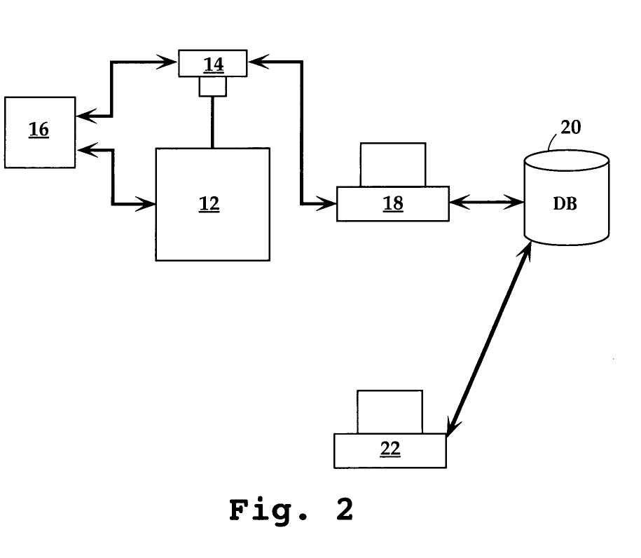

Referring to FIG. 2, a system for carrying out the preferred sequencing approach of the present invention according to embodiments of the invention is illustrated. A fluidic system 12 and detection system 14 are provided for collecting and imaging optical signals which are used to determine the sequences of the free ends of the cloned templates on each microbead in a flow cell. Delivery of fluids and collection of signals is controlled by computer 16 which may be of any suitable type. Further details of systems 12 and 14 and computer 16 are set forth in PCT/US98/11224 which is incorporated herein by reference.

As shown in FIG. 2, the detection system 14 is in communication with computer 18 where the computer-implemented aspects of the sequencing is performed. Computer 18 is preferably a workstation of the type available from Sun Microsystems. However, other suitable types of computers may also be used. Computer 18 is in communication with a database 20 which stores sequence data. Computer 18 may also perform the functions of computer 16, in which case computer 18 is also in communication with the fluidic delivery system. Another computer 22, which is in communication with database 20, may be used to perform quality control analysis.

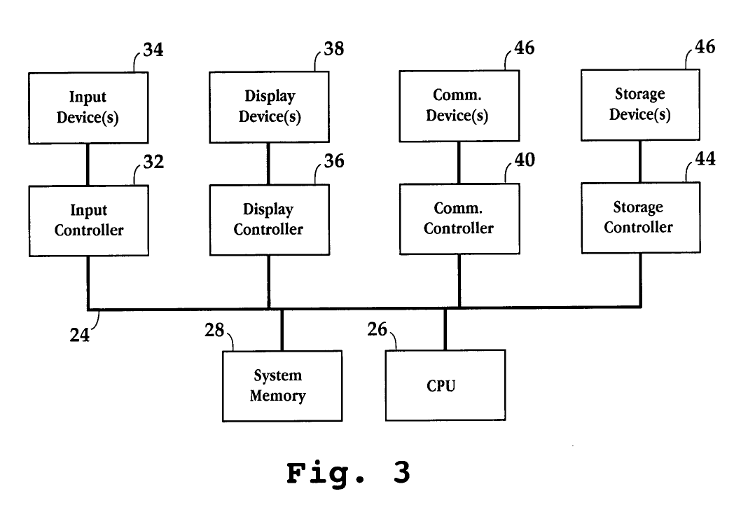

FIG. 3 is a functional block diagram showing various components of a computer system that may be used to implement computer 16, 18 and/or 22. As shown, this computer system includes bus 24 that interconnects central processing unit (CPU) 26, system memory 28 and several device interfaces. Bus 24 can be implemented by more than one physical bus such as a system bus and a processor local bus. CPU 26 represents processing circuitry such as a microprocessor, and may also include additional processors such as a floating point processor or a graphics processor. For computer 20, the CPU is preferably an E450 processor available from Sun Microsystems, Inc. System memory 28 may include various memory components, such as random-access memory (RAM) and read-only memory (ROM). Input controller 32 represents interface circuitry that connects to one or more input devices 34 such as a keyboard, mouse, track ball and/or stylus. Display controller 36 represents interface circuitry that connects to one or more display devices 38 such as a computer monitor. Communications controller 40 represents interface circuitry that connects to one or more communication devices 42 such as a modem or other network connection. Storage controller 44 represents interface circuitry that connects to one or more external and/or internal storage devices 46, such as a magnetic disk or tape drive, optical disk drive or solid-state storage device, which may be used to record programs of instructions for operating systems, utilities and applications which may include embodiments of programs that implement various aspects of the present invention.

It should be noted that FIG. 3 is merely an example of one type of system that may be used to implement computer 16, 18 and/or 20. Other suitable types of computers may be used as well, including computers with a bus architecture different from that illustrated in FIG. 3.

Various aspects of the sequencing process carried out on computer 18 may be implemented by a program of instructions (e.g., software). Similarly, the quality control functions performed by computer 20 may be implemented by software. Such software may be fetched by the computer CPU for execution. The software may be stored in a storage device 46 and transferred to RAM 28 when in use. Alternatively, the software may be transferred to the computer through a communication device such as a modem. More broadly, the software may be conveyed by any medium that is compatible with the computer. Such media may include, for example, various magnetic media such as disks or tapes, various optical media such as compact disks, as well as various communication paths throughout the electromagnetic spectrum including infrared signals, signals transmitted through a network including the internet, and carrier waves encoded to transmit the software.

As an alternative to software implementation, the above-described computer-implemented aspects of the invention may be implemented with functionally equivalent hardware using discrete logic components, one or more application specific integrated circuits (ASICs), digital signal processing circuits, or the like. Such hardware may be physically integrated with the computer hardware or may be a separate device which may be embodied on a computer card that can be inserted into an available card slot in the computer.

Thus, the above-described aspects of the invention can be implemented using software, hardware, or combination thereof. The diagrams and accompanying description provide the functional information one skilled in the art would require to implement a system to perform the functions required. Each of the functions may be implemented, for example, by software, functionally equivalent hardware, or a combination thereof.

IV. Principle of MPSS Analysis

Sequencing templates are “cloned” on microbeads by first generating a complex mixture of conjugates between the templates and oligonucleotide tags, where the number of different oligonucleotide tags is at least a hundred-fold larger than the number of templates. A sample of conjugates is taken that includes 1% of the total number of tags, thereby ensuring that essentially every template in the sample has a unique tag. The sample is then amplified by PCR, after which the tags are rendered single stranded and specifically hybridized to their complementary sequences on microbeads to form a “microbead” library of templates. Further description regarding the generation of such microbead-containing sequencing templates is set forth in PCT/US98/11224 which is incorporated herein by reference.

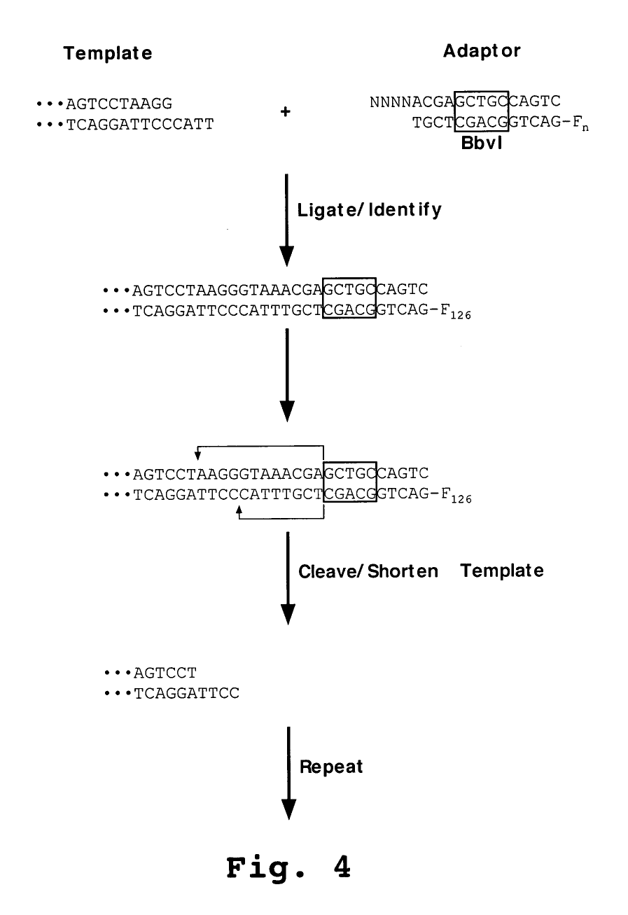

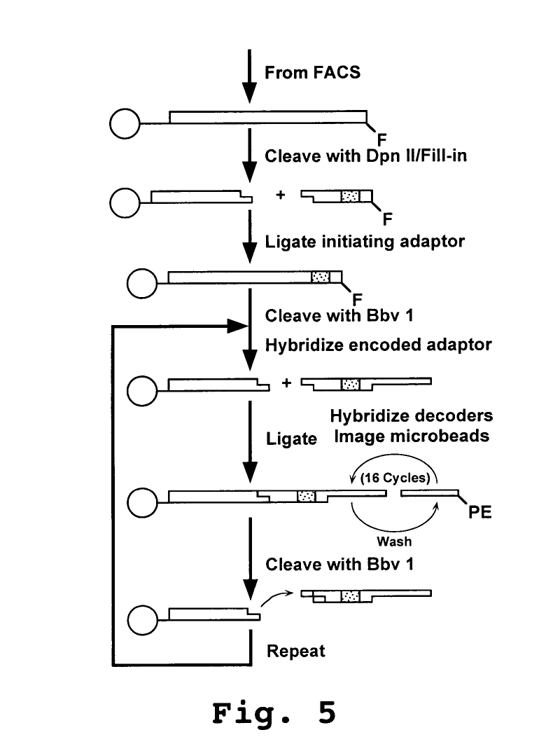

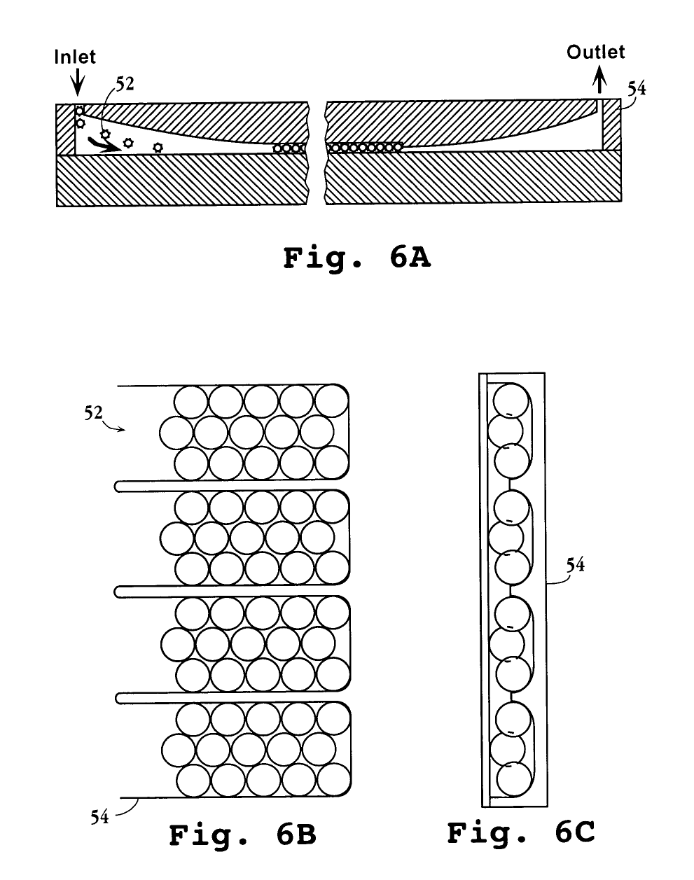

Referring to FIGS. 4 and 5, template sequences are determined by detecting successful adaptor ligations. A mixture of adaptors including every possible overhang is annealed to a target sequence so that only the one having a perfectly complementary overhang is ligated. Each of the 256 adaptors has a unique label, Fₙ, which may be detected after ligation. In FIG. 4, the sequence of the template overhang is identified by adaptor label F₁₂₆, which indicates that the template overhang is “TTAC.” The next cycle is initiated by cleaving with BbvI to expose the next four bases of the template. A signature is obtained by monitoring a series of such ligations on the surface of a microbead 52 whose position is fixed in a flow cell 54, as shown in FIGS. 6B and 6C.

The sequencing method takes advantage of a special property of a type IIs restriction endonuclease; namely, its cleavage site is separated from its recognition site by a characteristic number of nucleotides. Thus, a type IIs recognition site can be positioned in an adaptor so that after ligation, cleavage will occur inside the template to expose further bases for identification in the following cycle.

After microbeads loaded with fluorescently labeled (F) cDNAs are isolated by FACS, the cDNAs are cleaved with DpnII to expose a four-base overhang, which is then converted to a three-base overhang by a fill-in reaction. Fluorescently labeled (F) initiating adaptors containing BbvI recognition sites are ligated to the cDNAs in separate reactions, after which the microbeads 52 are loaded into flow cells 54, as shown in FIG. 6A. cDNAs are then cleaved with BbvI and encoded adaptors are hybridized and ligated. Sixteen phycoerythrin-labeled (PE) decoder probes are separately hybridized to the decoder binding sites of encoded adaptors and, after each hybridization, an image of the microbead array is taken for later analysis and identification of bases. The encoded adaptors are then treated with BbvI which cleaves inside the cDNA to expose four new bases for the next cycle of ligation and cleavage.

Preferably, cDNA templates on microbeads are initially cleaved by DpnII and the resulting ends converted to three-base overhangs, to be compatible with the initiating adaptors. Different initiating adaptors, whose type IIs restriction sites are offset by two bases, are ligated to two sets of microbeads to reduce signature losses from self ligation of ends of cDNAs whose cleavage with BbvI fortuitously exposes palindromic overhangs. Preferably, encoded adaptors (see Table 1) are used which permit the identification of four bases in each cycle of ligation and cleavage. In each cycle, a full set of 1024 encoded adaptors is ligated to the cDNAs, so that each microbead had four different adaptors attached, one for each position of the four-base overhang. The identity and ordering of nucleotides in the overhang of a template are encoded in the 10-mer decoder binding sites of the adaptors (lower case bases in Table 1) and are read off by specifically hybridizing in sequence each of sixteen decoder probes to the successfully ligated adaptors. The method continues with cycles of BbvI cleavage, ligation of encoded adaptors, and decoder hybridization and fluorescence imaging.

TABLE 1:

Sequences of encoded adaptors with four base overhangs
in bold and decoder binding sites in lower case.

Common strand:
5′-GACTGGCAGCTCGT
Encoded adaptors for detecting base 1:
5′-NNNAACGAGCTGCCAGTCcatttaggcg
5′-NNNGACGAGCTGCCAGTCctgattaccg
5′-NNNCACGAGCTGCCAGTCaccaatacgg
5′-NNNTACGAGCTGCCAGTCcgctttgtag
Encoded adaptors for detecting base 2:
5′-
NNANACGAGCTGCCAGTCggaacctgaa
5′-
NNGNACGAGCTGCCAGTCtgtgcgtgat
5′-NNCNACGAGCTGCCAGTCaccgacattc
5′-NNTNACGAGCTGCCAGTCattcctcctc
Encoded adaptors for detecting base 3:
5′-NANNACGAGCTGCCAGTCcgaagaagtc
5′-NGNNACGAGCTGCCAGTCtggtctctct
5′-NCNNACGAGCTGCCAGTCtagcggactt
5′-NTNNACGAGCTGCCAGTCggcgataact
Encoded adaptors for detecting base 4:
5′-ANNNACGAGCTGCCAGTCgcatccatct
5′-GNNNACGAGCTGCCAGTCcaactcgtca
5′-CNNNACGAGCTGCCAGTCcacagcaaca
5′-TNNNACGAGCTGCCAGTCgccagtgtta

To collect signature data, a microbead 52 must be tracked through successive cycles of ligation, probing, and cleavage, a condition which is readily met by using the flow cell shown in FIG. 6 or equivalent device which constrains the microbeads to remain in a closely packed monolayer. In one implementation, the flow cell was fabricated by micromachining a glass plate to form a grooved chamber for immobilizing microbeads in a planar array. Microbeads are held in the flow cell during application of reagents by a constriction in the vertical dimension of the chamber adjacent to the outlet.

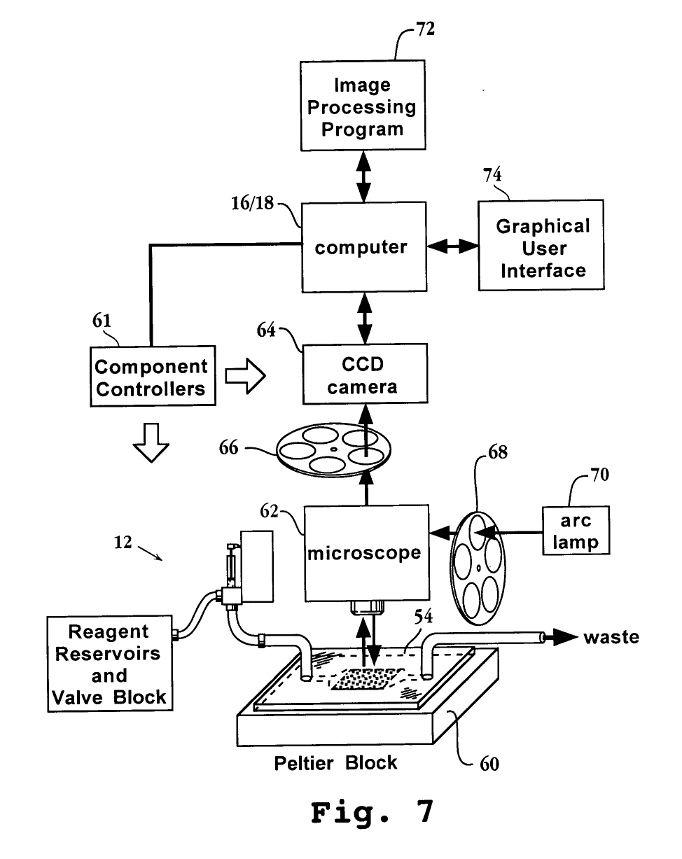

FIG. 7 is a schematic illustration detection system 14, and a computer which performs the functions of computers 16 and 18. In particular, the computer is adapted to collect and image fluorescent signals from the microbead array. Flow cell 54 and portions of fluidic delivery system 12 are also shown. Flow cell 54 resides on a peltier block 60 and is operationally associated with fluidic and detection systems 12 and 14 so that delivery of fluids and collection of signals is under control of the computer. Component controllers 61 interface between the computer and systems 12 and 14 to facilitate the control of these systems.

Preferably, optical (e.g., fluorescent) signals are collected by microscope 62 and are imaged onto a solid state imaging device such as a charge coupled device (CCD) 64 which is capable of generating a digital representation of the microbead array with sufficient resolution for individual microbeads to be distinguished.

For fluorescent signals, detection system 14 usually includes a band pass filter for the optical signal emitted from microscope 62 and a band pass filter for the excitation beam generated by light source (e.g., arc lamp) 70, as well as other standard components. The band pass filter for the optical signal may be carried, along with other band pass filters, on a filter wheel 66. Similarly, the band pass filter for the excitation signal may be carried on a filter wheel 68. A conventional fluorescent microscope is preferred which is configured for epiillumination. There is a great deal of guidance in the art for selecting appropriate fluorescence microscopes, e.g., Wang and Taylor, editors, Fluroescence Microscopy of Living Cells in Culture, Parts A and B, Methods in Cell Biology, Vols. 29 and 30 (Academic Press, New York, 1989).

An image processing program 72 running on computer 16/18 is preferably used to track positions of, and monitor fluorescent signals from, individual microbeads through successive hybridizations of decoder probes and through successive cycles of ligation and cleavage. Software running on the computer provides a graphical user interface (GUI) 74 for facilitating control of the fluidic and detection systems and interaction with the image processing program. In the embodiment of FIG. 7, GUI 74 also provides the tools for facilitating the computer-implemented sequencing in accordance with the invention.

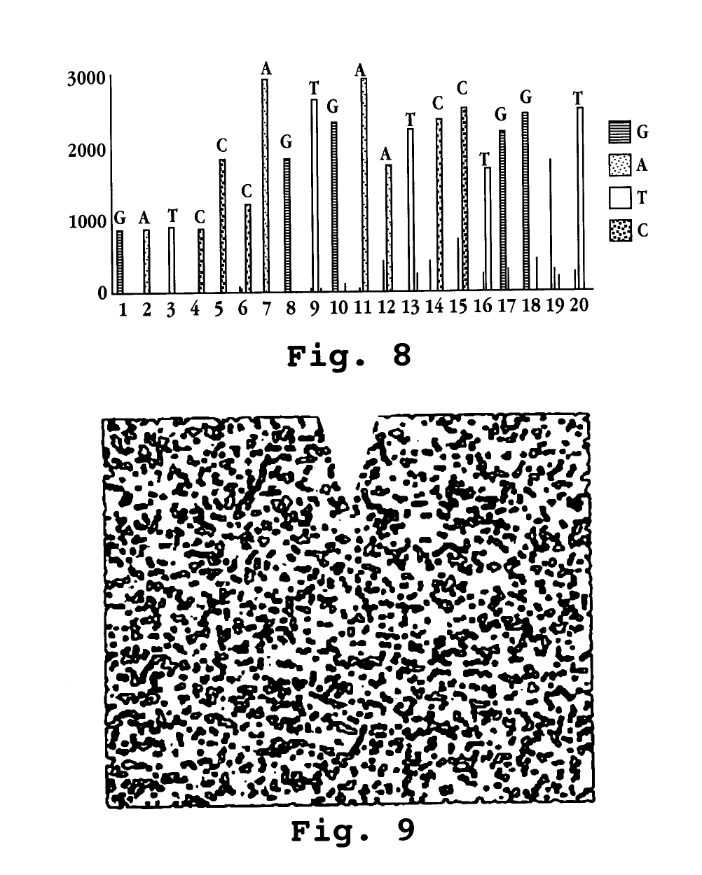

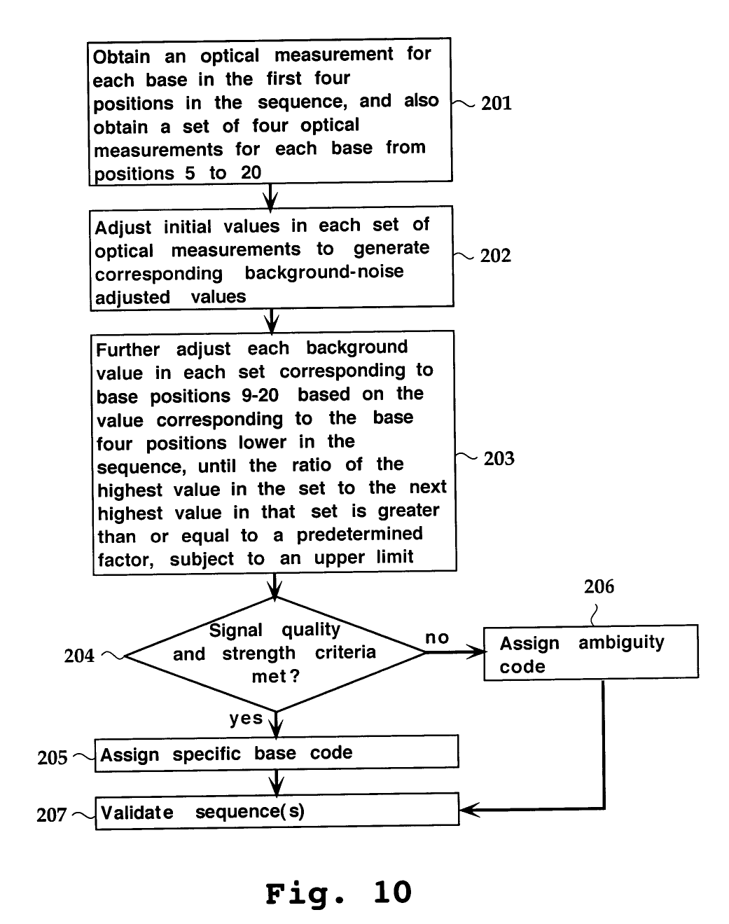

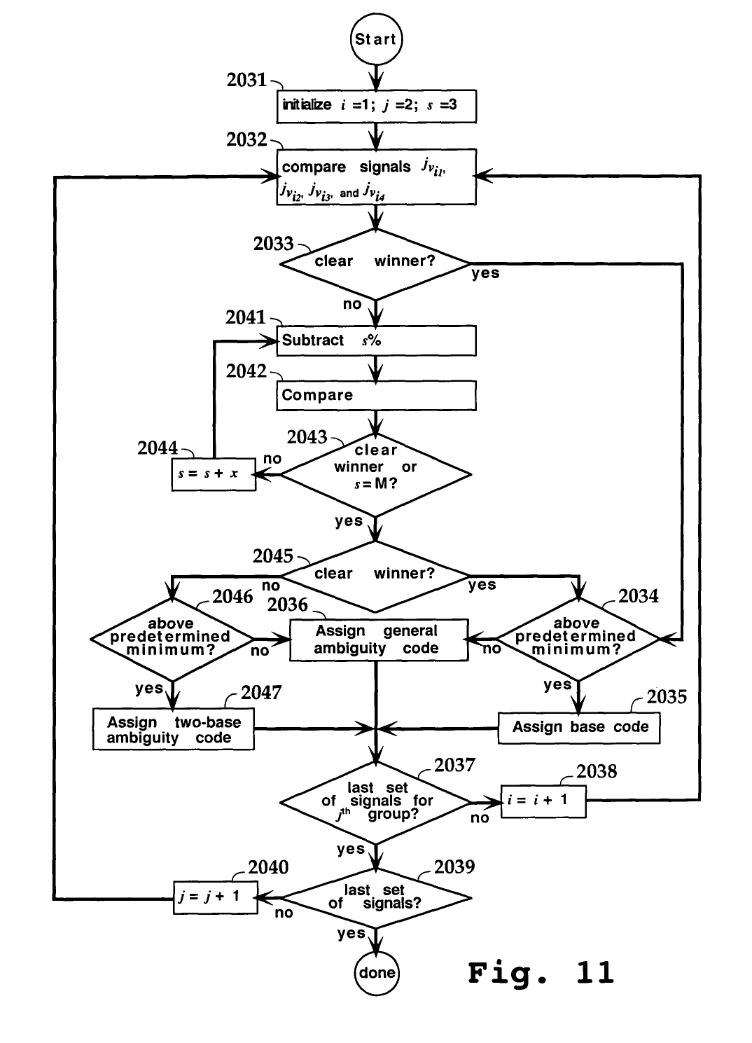

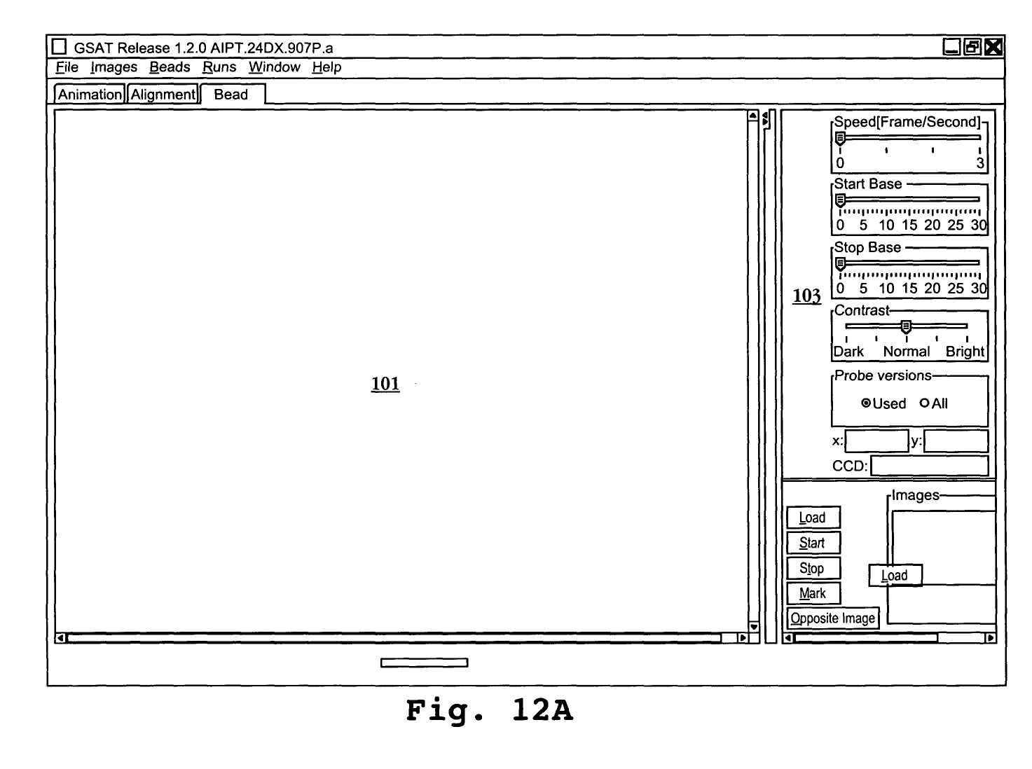

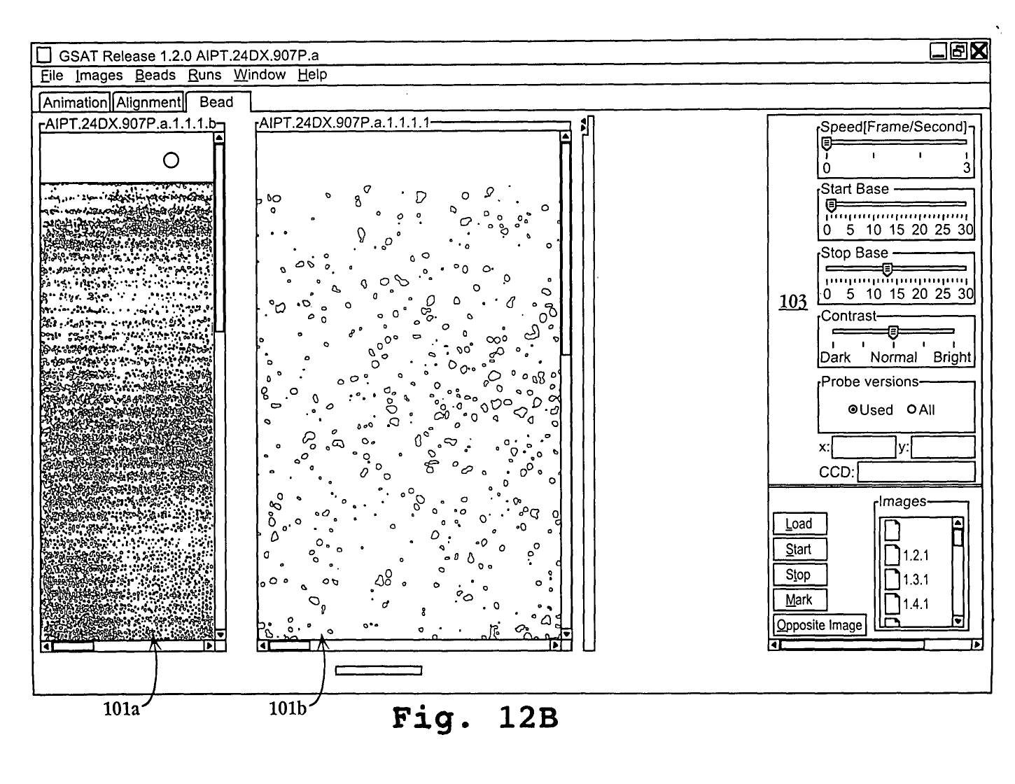

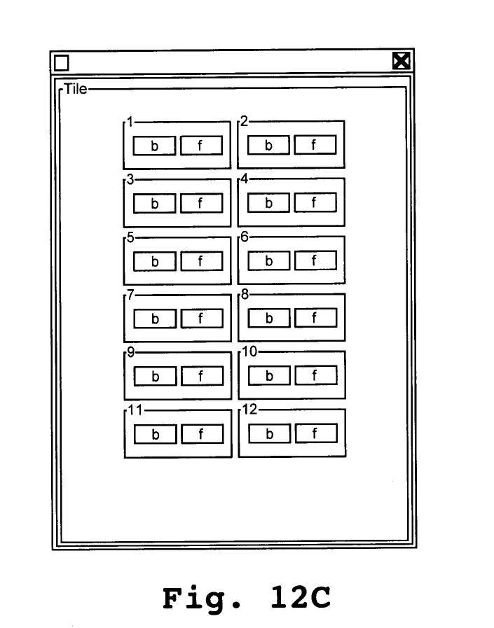

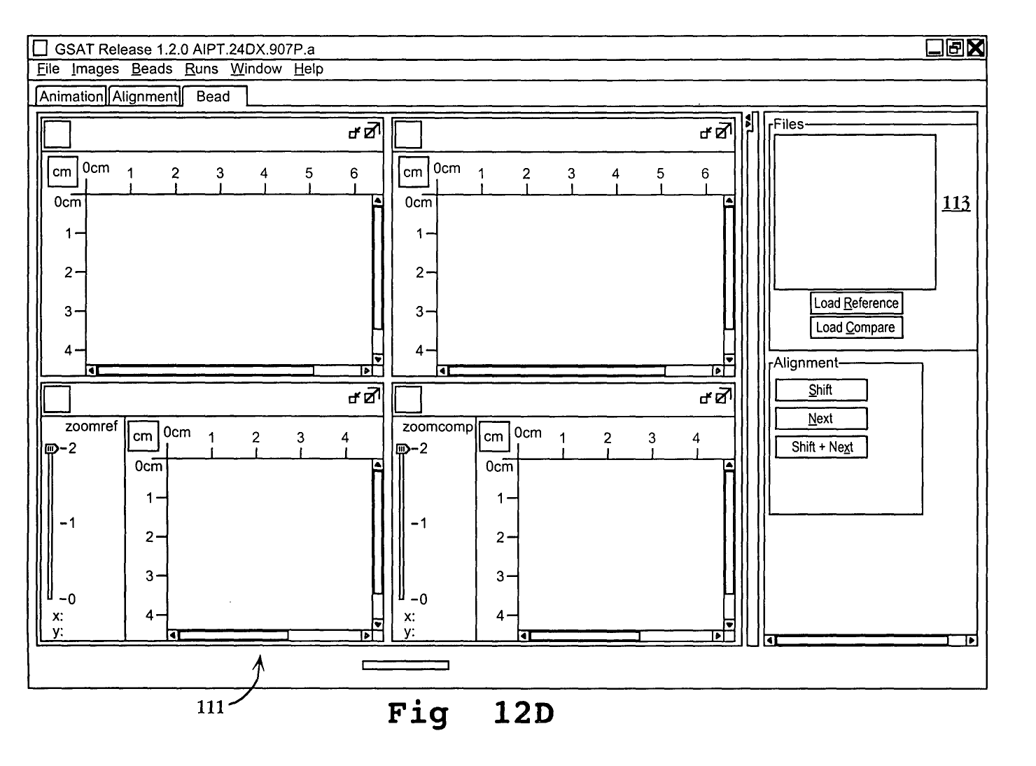

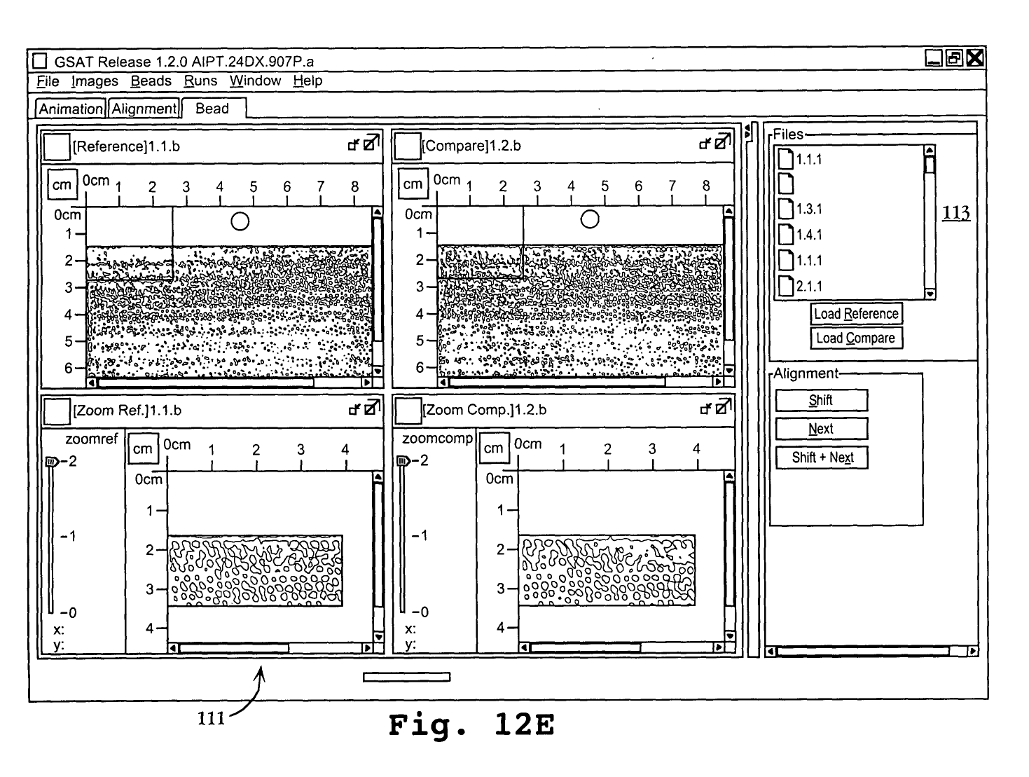

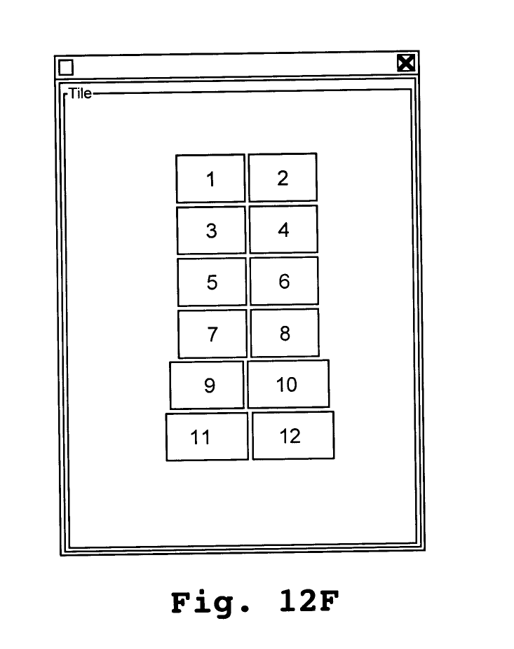

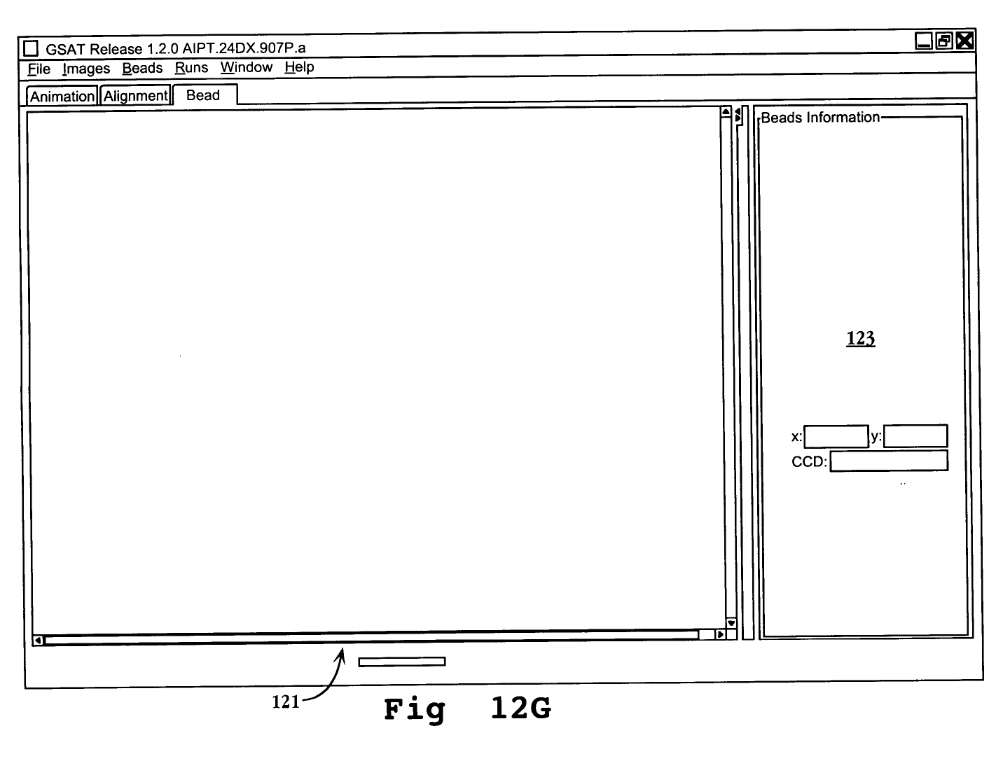

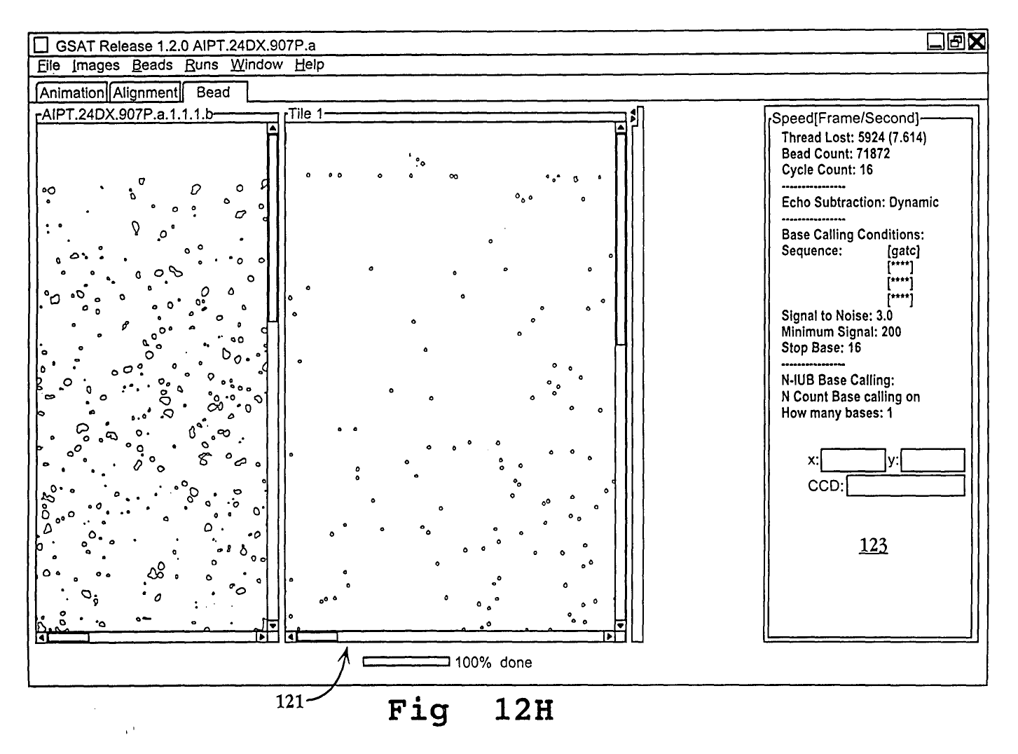

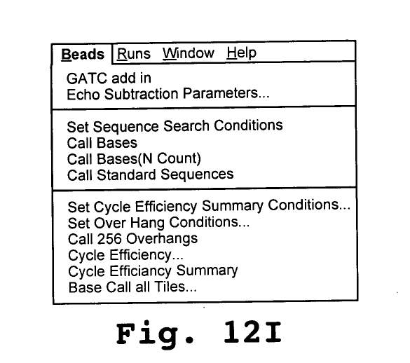

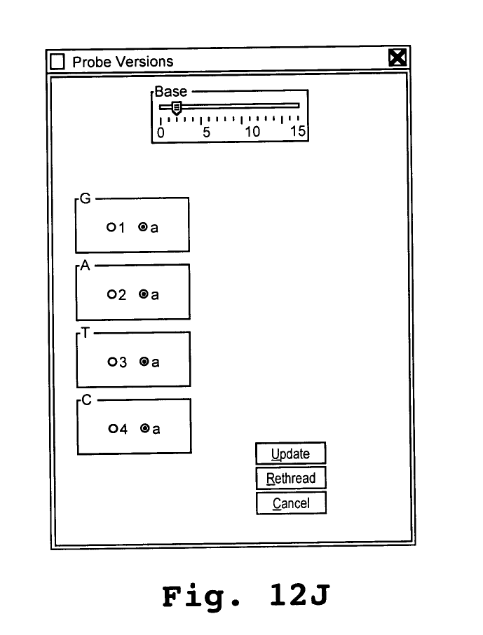

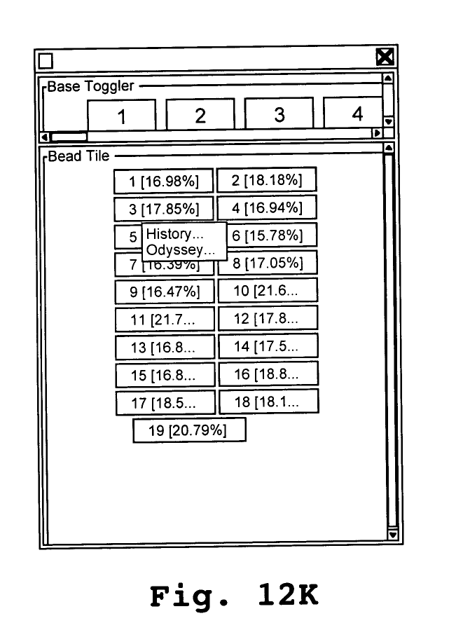

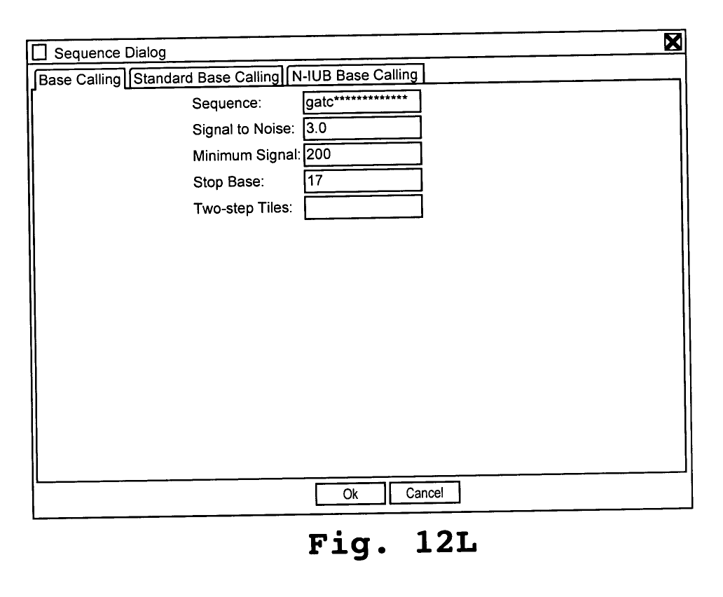

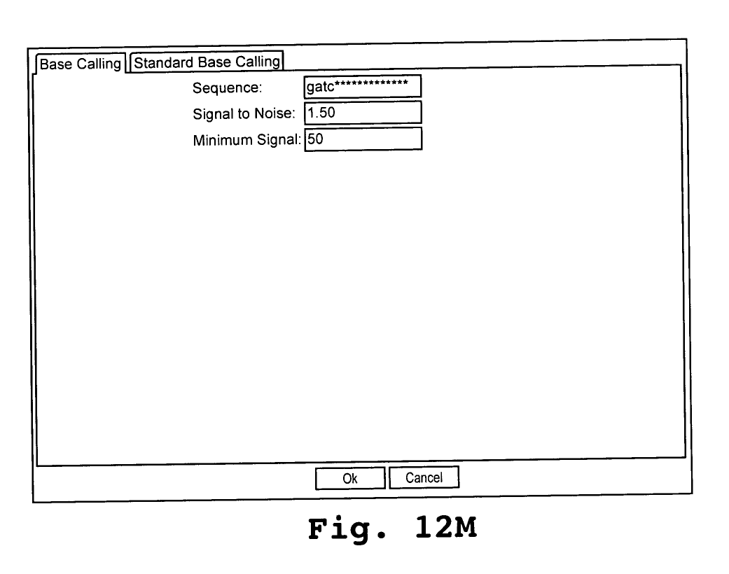

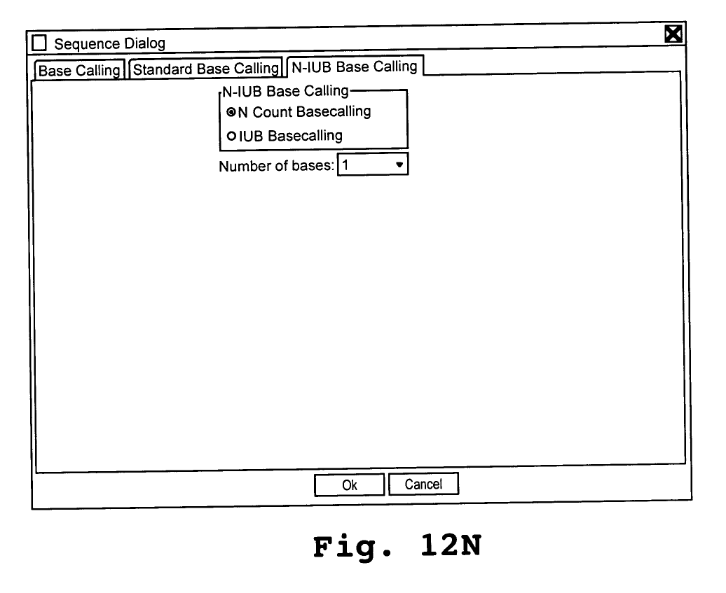

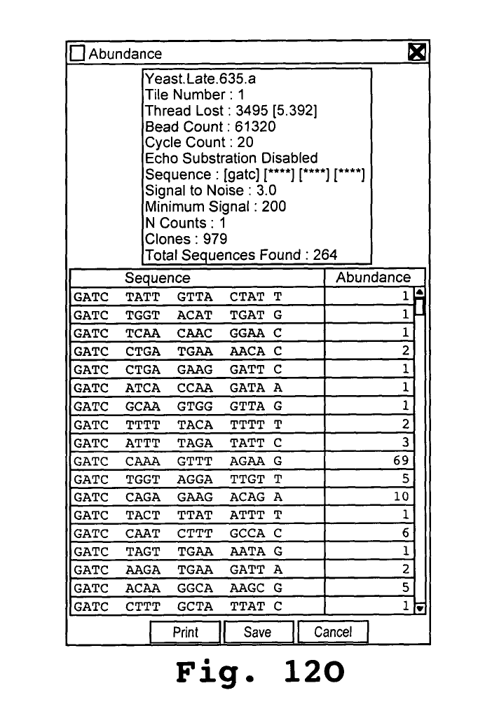

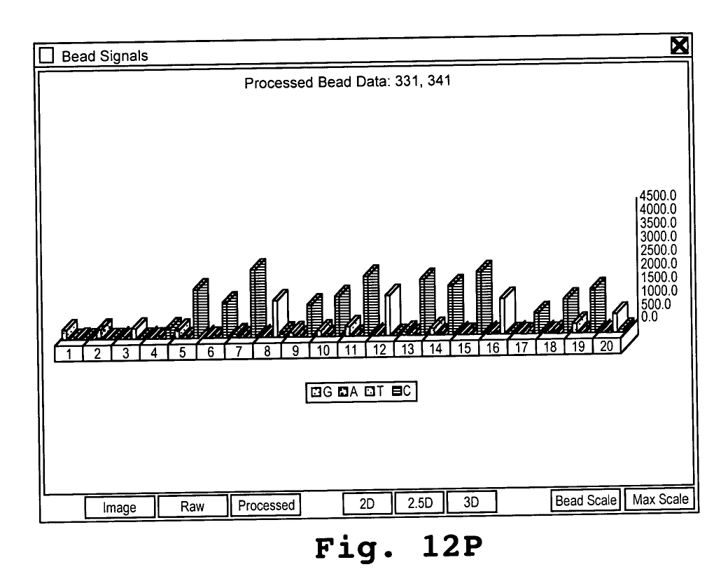

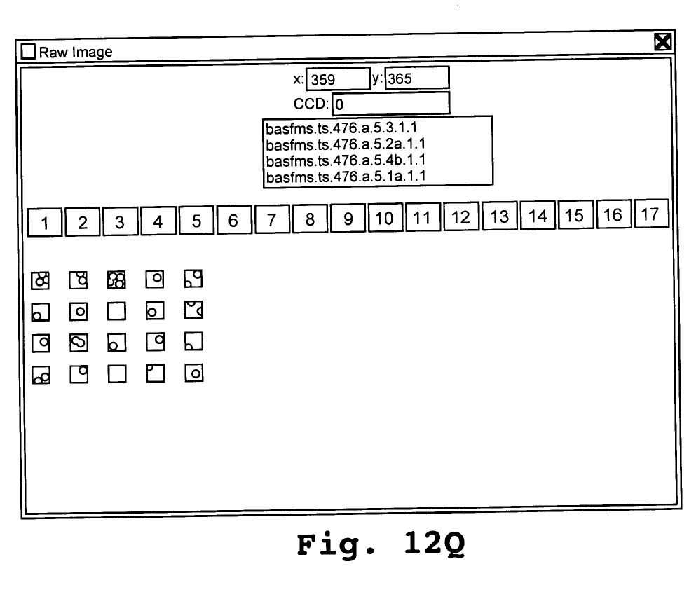

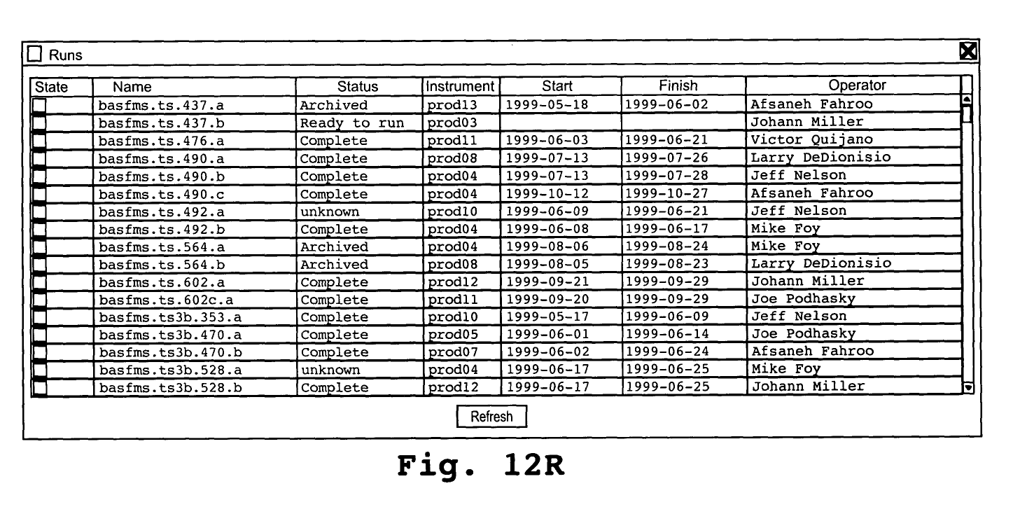

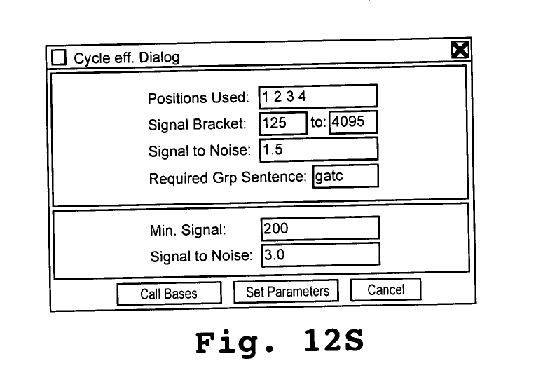

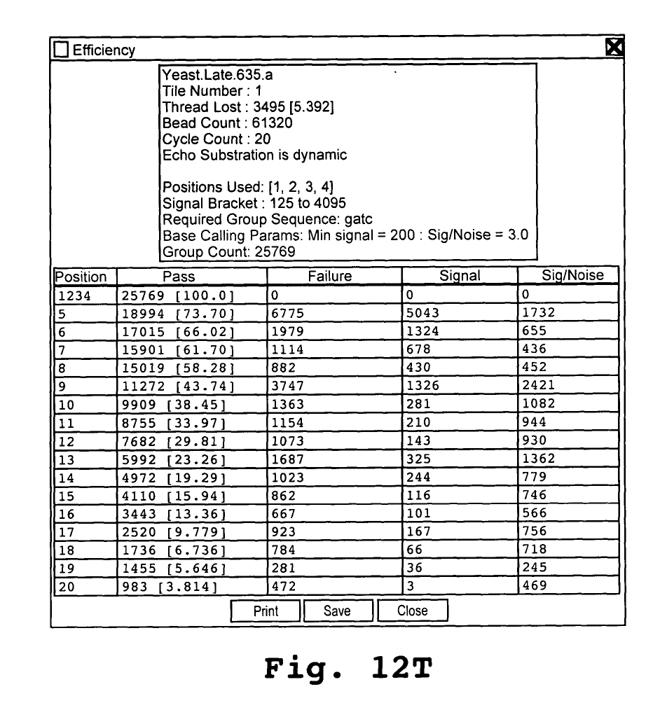

GUI 74 includes a microbead array display and a color-coded bar graph of the base calls for each base position in the analyzed sequence, as shown in FIGS. 8 and 9. As shown in the bar graph of FIG. 8, false color images of the microbead array display base calls in a color-coded format for any base position, and for each twenty-base signature a collection of 65 separate fluorescent signals are collected for every microbead in the flow cell. Further details of the base and signature calling algorithm are described below with reference to FIGS. 10 and 11, and GUI 74 is explained in more detail below with reference to FIGS. 12A through 12P and FIGS. 13A and 13B.

V. Experimental Protocol

1. Construction of Oligonucleotide Tag and Anti-Tag Libraries, in vitro Cloning, and Formation of Microbead Libraries

Reagents and procedures used for in vitro cloning of cDNA templates on microbeads have been described elsewhere. [12] Briefly, a library of 32-mer anti-tags was synthesized by eight rounds of combinatorial addition of eight 4-mer subunits on glycidyl methacrylate microbead substrates (Bangs Laboratories). Approximately 10% of the anti-tags attached by a base-labile group were cleaved and used to construct a tag vector library into which cDNA derived from yeast or THP-1 cells was inserted to form tag-cDNA conjugate libraries. DNA was transformed into electro-competent E. coli TOP10 cells (Invitrogen), which were grown in liquid cultures. For the microbead libraries, samples of 160,000 clones each were grown in 50 ml liquid cultures, after which tag-cDNA vectors were purified and tagged cDNAs were amplified using flanking PCR primers, one of which was fluorescently labeled. Tags of the amplified DNA were rendered single stranded as described, [12] and 50 μg of the resulting mixture was combined with an aliquot of 16.7 million microbeads, each having about 106 copies of a single anti-tag, in a 100 μl reaction. The sample was incubated for 3 days at 72° C., after which the microbeads were washed twice and the 1% microbeads having the brightest fluorescent signals were sorted on a Cytomation MoFlo cytometer. Loaded, sorted microbeads were treated with T4 DNA polymerase in the presence of dNTP to fill in any gaps between the hybridized conjugate and the 5′ end of the anti-tag, after which the anti-tag was ligated to the cDNA by T4 DNA ligase.

2. Adaptors and Decoder Probes

Strands of 16 sets of 64 encoded adaptors (Table 1) were synthesized on an automated DNA synthesizer (from PE Biosystems) and separately combined with a common second strand to form double stranded adaptors each having a single stranded decoder binding site (lower case) and a Bbv I recognition site positioned so that cleavage occurs immediately beyond the adaptor's 4-base overhang. All 1024 adaptors were combined in Enzyme Buffer (EB) (10 mM Tris-HCl, 10 mM MgCl₂, 1 mM dithiothreitol, 0.01% Tween 20). 16 decoder probes were synthesized each having a sequence complementary to a different decoder binding site and a pyridyldisulfidyl R-phycoerythrin label (Molecular Probes) attached via a sulfosuccinimidyl 6-[3[2 pyridyldithio]propionamido]hexanoate cross-linker (Pierce) to an amino group (Clontech) attached through two polyethylene glycol linkers to the 5′ end of the decoder oligonucleotide. Sixteen decoder probes were made (10 nM decoder in System Buffer (SB), which consists of 50 mM NaCl, 3 mM MgCl₂, 10 mM Tris-HCl (pH 7.9), 0.1% sodium azide). To initiate sequencing reactions by BbvI cleavage at different positions along the cDNA templates offset by two bases, initiating adaptor 1 (5′-FAMssGACTGGCAGCTCGT, 5′-pATCACGAGCTGCCAGTC) and initiating adaptor 2 (5′-FAMssGACTGGCAGCAGTCGT, 5′-pATCACGACTGCTGCCAGTC) were synthesized, where “FAM” is 6-carboxyfluorescein (Molecular Probes), “s” is a polyethylene glycol linker (Clontech), and “p” is phosphate (Clontech). To block ligation of encoded adaptors to free tag complements on the microbeads, cap adaptor (5′-DGGGAAAAAAAAAAAAAA, 5′-xTTTTTTTTTT) was synthesized, where x is a thymidylic residue (Glen Research) attached in reverse orientation to prevent concatenation of adaptors.

3. Sequencing DNA on Microbeads

cDNAs on 2 million microbeads were digested with Dpn II (New England Biolabs) to provide a 5′-GATC overhang. After centrifugation and removal of the supernatant, the microbeads were treated with T4 DNA polymerase in the presence of 0.1 mM dGTP for 30 min at 12° C. to create three-base overhangs on the free ends of the attached cDNAs. The microbeads were divided into two parts and initiating adaptors 1 and 2 were separately ligated to different parts by combining 10⁶ microbeads in 5 μL of TE (10 mM Tris, 1 mM EDTA) and 0.01% Tween 20 with 3 μL 10× ligase buffer (New England Biolabs), 5 μL adaptor in EB (25 nM), 2.5 μL T4 DNA ligase (2000 units/μL), and 14.5 μL distilled water, and incubating at 16° C. for 30 minutes, after which the microbeads were washed 3× in TE (pH 8.0) with 0.01% Tween. After resuspension in TE with 0.01% Tween, 10⁶ microbeads of each part were loaded into separate flow cells where they were processed identically.

Reagents were pumped through the flow cells at a rate of 1 μL/min. SB was applied for 15 min at 37° C. and for 15 min at 25° C., after which cap adaptor (1 nmol/μL in EB, T4 DNA ligase (Promega) at 0.75 U/μL) was twice applied for 25 min at 16° C., first followed by SB for 10 min, Pronase wash (0.14 mg/mL Pronase (Boehringer) in phosphate buffered saline (Gibco) with 1 mM CaCl₂) for 25 min, and SB for 20 min, all at 37° C.; and second followed by SB for 10 min, Pronase wash for 25 min, Salt wash (SB with 150 mM NaCl) for 10 min, and SB for 10 min, all at 37° C. The microbeads were then imaged and positions in the flow cells recorded, after which three cycles of the following steps were carried out: BbvI (1 U/μL in EB with 1 nmol/μL of carrier DNA: 5′-AGTGAACCTCGTTAGCCAGCAATC) was applied for 30 min, followed by SB for 10 min, Pronase wash for 25 min, Salt wash for 10 min, and SB for 10 min, all at 37° C. Ligation mix (1 nmol/μL encoded adaptor, 0.75 U/μL T4 DNA ligase in EB) was twice applied for 25 min at 16° C., first followed by SB for 10 min, Pronase wash for 25 min, and SB for 20 min, and second followed by SB for 10 min, Pronase wash for 25 min, and SB for 10 min, all at 37° C. Kinase mix (0.75 U/μL T4 DNA ligase, 7.5 U/μL T4 polynucleotide kinase (New England Biolabs) in EB) was applied for 30 min at 37° C., followed by SB for 10 min, Pronase wash for 25 min, Salt wash for 10 min, and SB for 10 min, all at 37° C. SB was applied for 75 min at temperatures varying between 20° C. and 65° C., after which each decoder probe was successively applied for 15 min at 20° C., each application being followed by SB for 10 min at 20° C., microbead imaging with flow stopped, 100 mM dithiothreitol in SB for 10 min and SB alone for 10 min both at 37° C. Each cycle was completed by applying SB for 10 min, Pronase wash for 25 min, Salt wash for 10 min, all at 37° C., followed by SB for 10 min at 55° C. and for 15 min at 20° C.

4. Base and Signature Calling

The base and signature calling algorithm of the present invention will now be described with reference to the flow charts in FIGS. 10 and 11. In step 201 optical measurements having values ^{j}vᵢ₁, ^{j}vᵢ₂, ^{j}vᵢ₃ and ^{j}vᵢ₄ indicative of each nucleotide position i of each nucleotide group j, for i=1 through k and for j=1 through m, is obtained. In addition, a single optical measurement indicative of each of k nucleotides in a first nucleotide group (j=0) is obtained.

In this generalized nucleotide sequence structure, the number of nucleotides in a group, denoted by k, can range from 2 to 5, and the total number of groups of nucleotides excluding the first group, denoted by m, can range from 3 to 5. In addition, the m groups of k nucleotides need not be contiguous; even with gaps in between groups a good signature may still be obtained.

In the present implementation, k, m=4, with the m groups being contiguous. With those parameters, the sequence is 20 nucleotides, and the raw data for a signature of such a sequence consists of 16 sets of optical (e.g., fluorescence) measurements of 4 values each that correspond to the interrogation of each base position by decoder probes for A, C, G, and T, in each of four cycles, together with a single fluorescence value assigned to each nucleotide in the initial GATC overhang based on the signal from the initiating adaptor.

After the raw data was obtained, the initial values in each set of optical measurements were adjusted for system background noise, which can be the result of non-specific binding of probes, incomplete digestion from the previous ligation-cleavage cycle, or incomplete ligation from the current cycle. In the present implementation, this was done by computing the background noise for each signal set (taken as the average of the lowest three fluorescence values in that set) and subtracting that computed value from each of the four fluorescence values in the set to generate corresponding background adjusted values (step 202). Other methods of computing and compensating for background noise may also be used, including various statistical methods of modeling noise for the particular system used.

Next, in step 203, for every nucleotide position from i=1 through k in every nucleotide group from j=2 through m, values ^{j}vᵢ₁, ^{j}vᵢ₂, ^{j}vᵢ₃ and ^{j}vᵢ₄ (i.e., each set of optical measurements corresponding to nucleotides (k+1) through mk not counting the k nucleotides in the j=0 group) are further adjusted based on a corresponding values ^{j-1}vᵢ₁, ^{j-1}vᵢ₂, ^{j-1}vᵢ₃ and ^{j-1}vᵢ₄ (i.e., values for the base four positions lower in the sequence), until the ratio of the highest value in the present set to the next highest value in that set is greater than or equal to a predetermined factor n, subject to an upper limit. Thus, in the present implementation, starting with base position 9 (including the k nucleotides in the j=0 group), increasing fractions of the values at positions four lower, i.e., 5 for 9, 6 for 10, and so on, were subtracted from corresponding values at the higher positions until a single value at the higher position was obtained that was at least n times the next highest value. The iterative subtraction process of step 203 is subject to a maximum subtraction percentage M which is measured as a percentage of the unadjusted signal value. This step adjusts the values of positions 9 through 20 for carry-over signal due to inefficient cleavage of adaptors.

Next, in step 204 it is determined if certain criteria indicative of signal quality and relative signal strength are met. If so, the process proceeds to step 205 where a specific base code is assigned to the position corresponding to that signal set. Otherwise, an ambiguity code is assigned to that position in step 206. Following assignment, the sequence is validated in step 207.

The process of steps 203-206 are explained in more detail with reference to the flow chart of FIG. 11. At the start of the process, nucleotide base position variable i is initialized to 1, and nucleotide group variable j is initialized to 2 in step 2031. A subtraction percentage variable s is also initialized to some initial subtraction fraction or percentage (2% in the present implementation) at the start of the process in step 2031.

The process continues at step 2032 where background adjusted values ^{j}vᵢ₁, ^{j}vᵢ₂, ^{j}vᵢ₃ and ^{j}vᵢ₄ are compared. Note that with an initial 4 base overhang, k, m=4, and i initialized at 1 and j at 2, the first set of optical signals compared correspond to nucleotide position 9. If one of the signals has a value that is greater than the next highest value by the predetermined factor n, that signal is declared the winner in step 2033, and no further adjustment is necessary. The process then continues at step 2034, where it is determined if the highest value in the signal set is above a predetermined minimum value. If so, a specific base code corresponding to that highest signal value is made for that position in step 2035. Otherwise, a general ambiguity code is assigned in step 2036.

Following assignment in step 2035 or no assignment in step 2036, it is then determined if all sets of signals in the j^{th} group have been analyzed (step 2037). If not, nucleotide position variable i is incremented in step 2038 and the next set of signals in the j^{th} group are compared in step 2032. If all sets of signals in the j^{th} group have been analyzed and that is not the last nucleotide group/signal set, as determined in step 2039, j is incremented and i is reinitialized in step 2040, after which the first signal set of the next group is compared in step 2032. In the present implementation, where m, k=4, j is incremented every fourth time i is incremented.

For any given set of signals corresponding to nucleotide positions (k+1) through mk (i.e., positions 9 through 20 of the total sequence in the present implementation), if the condition in step 2033 is not satisfied, an iterative subtraction process is performed. The subtraction process begins at step 2041 by subtracting s % of the background adjusted value of the signal four positions lower from the corresponding background adjusted signal value at the higher position. That is, s % of each of ^{j-1}vᵢ₁, ^{j-1}vᵢ₂, ^{j-1}vᵢ₃ and ^{j-1}vᵢ₄ is respectively subtracted from ^{j}vᵢ₁, ^{j}vᵢ₂, ^{j}vᵢ₃ and ^{j}vᵢ₄. For example, s % of the value of each signal at position 5 is subtracted from the value of the corresponding signal at position 9, and so on.

Another comparison is then made in step 2042 amongst the values in the higher set to determine if the highest value in that set is greater than the next highest value by at least the predetermined factor n, or if s=M which represents a predetermined maximum subtraction percentage. If neither of these conditions are satisfied, as determined in step 2043, the subtraction percentage variable s is increased by x in step 2044, and the process returns to step 2041 where (s+x) % of the background adjusted value of each signal four positions lower is subtracted from the corresponding signal value at the higher position. It should be noted that if additional subtraction iterations are needed, the subtraction is done on the signal values before any previous subtraction operations were performed. In the present implementation, x is 2.

This iterative subtraction loop of steps 2041 through 2044 repeats until one of the values in the present set is greater than the next highest value in that set by the predetermined factor n, or until the subtraction percentage s reaches the predetermined upper limit M, at which point the loop is exited. M=40 in the present implementation.

After the subtraction loop is exited, the algorithm continues at step 2045 where it is determined if the highest value in the present signal set is greater than the next highest value by at least the predetermined factor n. If so, the process proceeds to step 2034.

If the decision in step 2045 is “no,” the process continues at step 2046, where it is determined if both the highest and the next highest values in the signal set are above the predetermined minimum value. If so, a two-base ambiguity code corresponding to those two signals is assigned to that nucleotide position in step 2047. If not, a general ambiguity code is assigned in step 2036. Following either of steps 2047 or 2036, the algorithm continues to 2037. After all sets of signals have been analyzed, the process terminates.

In the present implementation, the predetermined factor n is 3. However, this value is exemplary only. In general, the predetermined factor n is empirically determined by calibrating the instrument on a test system, which may be an appropriate fully characterized set of sequences, preferably a sequenced genome. In the present implementation, the test system was yeast, as previously described. Thus, depending on the test system other suitable factors may be used. Typically, n will range from about 2 to about 5. Lower predetermined factors may lead to false positive base identification, while higher factors may result in the assignment of an ambiguity code when in fact the data was sufficiently conclusive to call a specific base.

Moreover, it should be noted that although the subtraction percentage was initially set at 2% and incremented by an additional 2% each time until an upper limit of 40% was reached, these values for s, x and M are exemplary only. Other values may be used for these variables of the iterative subtraction process.

In general, the setting of s is based on the initial ratio of the highest value in the signal value set presently being adjusted to the next highest value in that set. A lower s value is more appropriate when the initial ratio tends to be close to predetermined factor.

The setting of x generally involves a trade-off between precision and processing speed. In general, the lower x is set the more processing and iterations are required. However, setting x too high may decrease the precision of the process.

The setting of M is based on considerations of signal reliability. That is, M represents an upper limit of how much can be subtracted from a background adjusted signal value before the signal becomes unreliable. M may be based on signal-to-ratio characteristics. For purposes of this invention, it is believed that M should be set such that the highest background adjusted signal value in a set does not fall below 125% of the background value.

In the present implementation, the predetermined minimum value is twice the background noise level. However, this value is exemplary only. In general, the predetermined minimum value is a measure of a minimally reliable signal and is detector dependent. Based on this guideline, other predetermined minimum values may be used. In general, the predetermined minimum value for a set should be at least 125% of the set's background noise level.

Regarding the assignment of a specific base code (step 2035) or two-base ambiguity code (step 2047), in the present implementation, a base code (A, C, G, or T) corresponding to the highest signal value in the set was assigned to a position if the highest signal value was at least three times the next highest signal value in the set, and the highest value was above the predetermined minimum value. If the former condition was not met but the predetermined minimum value was satisfied for both the highest and next highest signal values, then a two-base ambiguity code (R, Y, M, K, S, or W) was called. If neither condition was met, then a general ambiguity code can be assigned in step 2036 indicating that the data is insufficient to even call a two-base ambiguity code. Certain criteria may be established to reject signatures having more than a certain number of ambiguity codes.

Returning now to FIG. 10, signature validation is performed in step 206. This may be done by checking the sequence in any suitable manner, such as by comparing the signatures against an appropriate sequence database. For example, in the present implementation, signatures were searched for homology in three yeast databases using the National Center for Biotechnology Information (NCBI) BLASTN ver. 2.0 [14] with default parameters, unless an ambiguous base was present in the signature. In the latter case, BLASTN was used with the word size parameter reset to 7. The SGD open reading frame DNA database [15] was searched first and a match was recorded if at least 16 consecutive bases matched those of a database sequence. If no matches were found for a signature, the NCBI yeast genomic database was then searched, and if still no matches were recorded, the NCBI non-redundant DNA database, nt, was searched.

5. Cell Culture

Saccharomyces cerevisiae strain S288C (ATTC No. 204508) was grown as described. [17] Briefly, strain S288C was grown with orbital shaking at 30° C. in YPD media. Early and late log phase cultures were harvested at densities of A₆₀₀=0.6 and A₆₀₀=3.2, respectively. Cells were disrupted by repeated vortexing in the presence of lysis buffer (Novagen) containing 500 μm glass beads (Sigma), after which mRNA was purified form the lysate using a Straight A's mRNA isolation system (Novagen). THP-1 cells (ATCC No. TIB-202) were grown in D-MEM/F12 media supplemented with 10% heat-inactivated fetal bovine serum and induced by PMA and LPS treatment as described elsewhere. [12]

VI. Graphical User Interface (GUI) and Software for Base and Signature Calling Algorithm

In accordance with aspects of the invention, a Genomic Sequence Analysis Tool (GSAT), embodied in software, is used for quality assurance of a MPSS run. The GSAT includes a GUI through which the user may interact with the base calling algorithm. Such interaction may include, for example, inputting various run parameters, checking the state of a run, analyzing a run, etc. For example, a user may check the state of a run at each enzymatic cycle by examining probe images, checking alignments, checking base calling functions, etc. to determine if there are any problems before proceeding to the next cycle. If there are problems, then the hybridization reaction can be repeated, in which case the quality assurance check can be exercised again.

The GUI includes a suite of menus, control buttons, status indicators and tabbed panels, which enable the user to access and interact with various aspects of the program. The tabbed panels enable the user to switch between different GSAT modes, including an “Animation” mode, an “Alignment” mode, and a “Bead” mode. When a particular mode is selected, the control buttons associated with that mode are enabled.

The main window of the Animation mode is illustrated in FIGS. 12A and 12B. That window includes a display area 101 shown with no data in FIG. 12A but which may be used to display animated images of a sequencing-containing bead array, as illustrated in FIG. 12B. In the illustrated embodiment, two images of opposite type are displayed: a back-lit image 101a and a fluorescent image 101b. The main window of the Animation mode further includes a gauge panel 103, which has controls for image caching speed, bases at which to start and stop viewing animating probe images, image contrast (when image is not animated), and probe version. The gauge panel also shows the x- and y-coordinates of the current position of the cursor on the imaged bead array and the CCD count. Through interaction with the gauge panel, the user is able to see a probe's image list, including which images are used for spatially locating individual beads in the array. A tile selection window, illustrated in FIG. 12C, may be opened up on top of the Animation mode main window and used to select a tile (i.e., an imaged section of the bead-containing flow cell) for viewing. The “b” and the “f” represent back-lit and fluorescent respectively.

The main window of the “Alignment” mode, illustrated in FIGS. 12D and 12E. Through this window the user can access functions to align shifted images to show bead movement based on a comparison with a reference image. Such images may be loaded into a display area 111, as illustrated in FIG. 12E, using functions provided in a panel window 113. The display area 111 is partitioned into four windows: a window for holding a reference image, a window for holding a comparison image, a window for zooming the reference image and a window for zooming the comparison image. A tile selection window, illustrated in FIG. 12F, may be opened up on top of the Alignment mode main window and used to select a tile for viewing.

The main window of the “Bead” mode, illustrated in FIGS. 12G and 12H, enables the user to perform the various functions listed in the pull-down menu shown in FIG. 12I. The main Bead window includes a display area 121 shown with no data in FIG. 12G and with two images displayed in FIG. 12H. The two displayed images may be used to illustrate a bead array in different forms. For example, the image on the right shows “raw” bead data and the image on the left shows “processed” bead data. The main Bead window also includes a panel 123, which may be located to the right of the display area 121, as illustrated in FIGS. 12G and 12H. This panel displays a variety of bead history information, including various parameters that have been previously entered.

For any given run many hybridization reactions may be repeated, producing different versions of probe images. GSAT allows a user to choose any probe version to spatially relocate individual beads in an array. This is done through the “Images” pull-down menu on the main menu. A dialog box, as illustrated in FIG. 12J, allows a user to select a base to investigate by using a slider control. An indicator indicates which of two versions for each of the probes G, A, T and C is currently being used. In the illustrated embodiment, “1” refers to the original and “a” refers to a re-probe, i.e., a probe which has been rehybridized.

Base calling functions are enabled when the “Bead” tab is selected. A suite of functions are available in this category including (1) calling bases to check for sequences and their abundance, (2) checking cycle efficiency, and (3) continuing to the next cycle or re-probing the current one. The suite of functions may include those shown in FIG. 12I.

To perform one of the base calling functions, a tile (i.e., an imaged section) of a flow cell is first selected. FIG. 12K shows a screen from which one of nineteen tiles can be selected. The bracketed number next to each tile number represents bead or thread loss percentage. The “Base Toggler” function enables the user to view the highest signal at a particular base position. For example, to see which signal is the highest at the first base position, the user would click the “1” button.

After a tile is loaded, GSAT applies an echo subtraction parameter in accordance with a selected user option. The user may choose to manually input the echo subtraction value, allow GSAT to automatically determine the optimal echo subtraction value, or allow GSAT to dynamically determine echo subtraction while doing the base calling.

A function is also available for obtaining a history of a particular tile, providing information such as how many pixels were shifted in the x and y directions and thread loss for a particular probe of a particular cycle. “Odyssey” shows how many times a tile has been threaded. It is similar to “History” but “Odyssey” also keeps track of which probe versions were used to generate the thread file.

Setting the sequence search conditions can be done from the “Bead” pull-down menu. Using dialog boxes, as illustrated in FIG. 12L, the user inputs various requested information to carry out the processing desired. The Standard Base Calling panel (FIG. 12M) enables the user to find standard sequences that were used. The N—IUB Base Calling panel (FIG. 12N) allows for one or more failures and ambiguity codes in the base calling algorithm.

After calling a base sequence, a sequence-abundance dialog box, as shown in FIG. 120, appears if there are matched sequences. Sorting by sequences or abundance may be accomplished by clicking on the appropriate header. Beads for a particular sequence may be determined by selecting a sequence from the abundance table. Data for a particular bead of interest may be conveniently obtained by clicking on a particular bead in a bead array displayed in area 121. The processed data (after echo subtraction) for that bead may then be presented in graphical form, such as a color-coded bar graph illustrated in FIG. 12P, which shows the base calls for each base position in an analyzed sequence. A plurality of different selectable functions, which may be in the form of graphical push buttons, are displayed near the data graph. The user may select a type of data to view, e.g., image, raw, or processed by selecting the appropriate button. The type of function associated with each push button is conveniently displayed on the button.

A display of a bead's raw image data is shown in FIG. 12Q. As shown in FIG. 12Q, a bead's raw image data includes GATC probe images that allow a user to verify whether the base calling was done correctly. Within each column of images there should be only one that has the highest CCD value at the bead's x, y coordinate.

Base calling can also be done for standard sequences and 256 overhang.

From the “runs” pull-down menu, the user may obtain a list of runs (FIG. 12R) entered into the MPSS database 20, which may be sorted in a variety of ways, e.g., by name, run status, the instrument on which the run is performed, start date, finish date, etc., by clicking the corresponding column header. The status field indicates the status of a particular run, and by clicking on that field, a user may obtain more detailed information regarding the run's progress. A pop-up dialog box appears showing a detailed list of what actions have been taken for the run, e.g., whether ftp processes to transfer probe images have started or whether threading has occurred. To select a run for quality control analysis, the user may click on any field of that run except Status.

The program also allows the user to check cycle efficiency using a dialog box (FIG. 12S), and to display the results of such a check (FIG. 12T).

Signature accuracy was assessed by constructing cDNA libraries from mRNA extracted from early and late log phase yeast cultures, and subjecting them to MPSS analysis (see Table 2). Of the 269,093 signatures called by the data processing algorithm, more than ninety percent were identified in public yeast databases, which is comparable to a similar measurement by serial analysis of gene expression (SAGE). [13] These results not only provide evidence of the accuracy of MPSS analysis, but also provide strong validation of the in vitro cloning technique. Without significantly pure populations of templates on the surfaces of the microbeads, few if any signatures would have been obtained.

TABLE 2
Accuracy of MPSS signatures for yeast.

| Log | Clones | Signatures | |
|---|---|---|---|
| Phase | Sequenced | Identified | Percent |
| Early: | 126,678 | 115,685 | 91% |
| Late: | 142,415 | 127,934 | 90% |
| Totals: | 269,093 | 243,619 | |

It should be readily apparent from the foregoing description that the present invention provides a novel sequencing approach that combines non-gel-based signature sequencing with in vitro cloning of millions of templates on separate microbeads. The sequencing approach includes a base calling algorithm which may be implemented with a program of instructions running on a computer. The program includes a GUI for allowing a user to interact with the algorithm.

While embodiments of the invention have been described, it will be apparent to those skilled in the art in light of the foregoing description that many further alternatives, modifications and variations are possible. The invention described herein is intended to embrace all such alternatives, modifications and variations as may fall within the spirit and scope of the appended claims.

What is claimed:

1. A method for determining a signature of a nucleotide sequence, comprising:
(a) obtaining optical measurements having values ^{j}vᵢ₁, ^{j}vᵢ₂, ^{j}vᵢ₃, and ^{j}vᵢ₄ indicative of each nucleotide in each of a j^{th} group of nucleotide positions i, for i equal 1 through k and for j equal 1 through m;
(b) for every group of nucleotide positions from j equal 2 through m, and every position from i equal 1 through k, adjusting the values ^{j}vᵢ₁, ^{j}vᵢ₂, ^{j}vᵢ₃, and ^{j}vᵢ₄ by repeatedly subtracting from each a first predetermined fraction of ^{j-1}vᵢ₁, ^{j-1}vᵢ₂, ^{j-1}vᵢ₃, and ^{j-1}vᵢ₄, respectively, until the ratio of the highest value in the set of ^{j}vᵢ₁ through ^{j}vᵢ₄, to the next highest value in the same set is greater than or equal to a predetermined factor, or until the repeatedly subtracted fractions have a sum equal to a second predetermined fraction; and
(c) generating a base call for position i in the j^{th} group based on results of the adjusting in (b).

2. The method of claim 1, wherein said base call generating (c) comprises
assigning a base code corresponding to the highest value to position i in the j^{th} group whenever the highest value is greater than or equal to a predetermined minimum value and the ratio of the highest value in the set of ^{j}vᵢ₁ through ^{j}vᵢ₄, to the next highest value in the same set is greater than or equal to the predetermined factor, and
assigning a two-base ambiguity code corresponding to the highest value and the next highest value whenever the ratio is less than the predetermined factor and the highest value and the next highest value are each greater than or equal to the predetermined minimum value.

3. The method of claim 2, further comprising rejecting the signature whenever the number of ambiguity codes assigned is greater than one.

4. The method of claim 2, wherein said obtaining (a) comprises adjusting values ^{j}vᵢ₁, ^{j}vᵢ₂, ^{j}vᵢ₃, and ^{j}vᵢ₄, for i equal 1 through k and for j equal 1 through m, for background noise.

5. The method of claim 4, wherein the background noise is computed as the average of the lowest three of ^{j}vᵢ₁, ^{j}vᵢ₂, ^{j}vᵢ₃, and ^{j}vᵢ₄, and wherein the computed background noise is subtracted from each of ^{j}vᵢ₁, ^{j}vᵢ₂, ^{j}vᵢ₃, and ^{j}vᵢ₄.

6. The method of claim 2, wherein the groups of positions, j=1 through m, are contiguous.

7. The method of claim 2, wherein m=3, 4 or 5.

8. The method of claim 7, wherein m=4.

9. The method of claim 2, wherein k=1, 2, 3, 4 or 5.

10. The method of claim 9, wherein k=2, 3 or 4.

11. The method of claim 10, wherein k=4.

12. The method of claim 2, wherein the predetermined factor is between about 2 and about 5.

13. The method of claim 4, wherein the predetermined minimum value is greater than 125% of the background noise.

14. The method of claim 2, wherein the first predetermined fraction is 1/50.

15. The method of claim 4, wherein the second predetermined fraction is set such that the highest value does not fall below 125% of the background noise.

16. An apparatus for determining a signature of a nucleotide sequence, comprising:
(a) a storage medium that stores a plurality of sets of digital signal values ^{j}vᵢ₁, ^{j}vᵢ₂, ^{j}vᵢ₃, and ^{j}vᵢ₄ indicative of each nucleotide in each of a j^{th} group of nucleotide positions i, for i=1 through k and for j equal 1 through m; and
(b) a processor in communication with the storage medium to:
(i) adjust the values ^{j}vᵢ₁, ^{j}vᵢ₂, ^{j}vᵢ₃, and ^{j}vᵢ₄, for every nucleotide position from i equal 1 through k in every group of nucleotide positions from j equal 2 through m, by repeatedly subtracting from each a first predetermined fraction of ^{j-1}vᵢ₁, ^{j-1}vᵢ₂, ^{j-1}vᵢ₃, and ^{j-1}vᵢ₄, respectively, until the ratio of the highest value in the set of ^{j}vᵢ₁ through ^{j}vᵢ₄, to the next highest value in the same set is greater than or equal to a predetermined factor, or until the repeatedly subtracted fractions have a sum equal to a second predetermined fraction, and
(ii) generate a base call for position i in the j^{th} group based on results of the adjusting in (i).

17. The apparatus of claim 16, wherein, to generate a base call for position i in the j^{th} group, the processor
assigns a base code corresponding to the highest value to position i in the j^{th} group whenever the highest value is greater than or equal to a predetermined minimum value and the ratio of the highest value in the set of ^{j}vᵢ₁ through ^{j}vᵢ₄, to the next highest value in the same set is greater than or equal to the predetermined factor, and
assigns a two-base ambiguity code corresponding to the highest value and the next highest value whenever the ratio is less than the predetermined factor and the highest value and the next highest value are each greater than or equal to the predetermined minimum value.

18. The apparatus of claim 17, further comprising a display in communication with the processor, wherein the processor renders a graphical representation of the digital signal values on the display upon user command.

19. The apparatus of claim 17, further comprising a display in communication with the processor, wherein the processor renders a graphical representation of a plurality of microbeads, each containing at least one copy of the nucleotide sequence, on the display upon user command.

20. A system for determining a signature of a nucleotide sequence, comprising:
(a) a processing and detection apparatus including an optical train operable to collect and convert a plurality of optical signals into corresponding digital signal values that comprise a plurality of sets digital signal values ^{j}vᵢ₁, ^{j}vᵢ₂, ^{j}vᵢ₃, and ^{j}vᵢ₄ indicative of each nucleotide in each of a j^{th} group of nucleotide positions i, for i=1 through k and for j equal 1 through m;
(b) a storage medium that stores ^{j}vᵢ₁, ^{j}vᵢ₂, ^{j}vᵢ₃, and ^{j}vᵢ₄; and
(c) a processor in communication with the storage medium and being operable to:
(i) adjust the values ^{j}vᵢ₁, ^{j}vᵢ₂, ^{j}vᵢ₃, and ^{j}vᵢ₄, for every nucleotide position from i equal 1 through k in every group of nucleotide positions from j equal 2 through m, by repeatedly subtracting from each a first predetermined fraction of ^{j-1}vᵢ₁, ^{j-1}vᵢ₂, ^{j-1}vᵢ₃, and ^{j-1}vᵢ₄, respectively, until the ratio of the highest value in the set of ^{j}vᵢ₁ through ^{j}vᵢ₄, to the next highest value in the same set is greater than or equal to a predetermined factor, or until the repeatedly subtracted fractions have a sum equal to a second predetermined fraction, and
(ii) generate a base call for position i in the j^{th} group based on results of the adjusting in (i).

21. The system of claim 20, wherein, to generate a base call for position i in the j^{th} group, the processor
assigns a base code corresponding to the highest value to position i in the j^{th} group whenever the highest value is greater than or equal to a predetermined minimum value and the ratio of the highest value in the set of ^{j}vᵢ₁ through ^{j}vᵢ₄, to the next highest value in the same set is greater than or equal to the predetermined factor, and
assigns a two-base ambiguity code corresponding to the highest value and the next highest value whenever the ratio is less than the predetermined factor and the highest value and the next highest value are each greater than or equal to the predetermined minimum value.

22. The system of claim 21, further comprising a program of instructions for execution by the processor to carry out (i) and (ii).

23. The system of claim 22, wherein the program of instructions is embodied in software.

24. The system of claim 22, wherein the program of instructions is embodied in hardware formed integrally or in communication with the processor.

25. The system of claim 22, further comprising a display and a graphical user interface presented on the display for enabling a user to display and manipulate data and results.

26. The system of claim 21, further comprising a data base, in communication with the processor, for storing sequencing information.

27. The system of claim 26, further comprising a second processor in communication with the data base for performing quality control analysis on the sequence signature.

28. A processor-readable medium embodying a program of instructions for execution by a processor for performing a method of determining a signature of a nucleotide sequence, the program of instructions comprising instructions for:
(a) obtaining optical measurements having values ^{j}vᵢ₁, ^{j}vᵢ₂, ^{j}vᵢ₃, and ^{j}vᵢ₄ indicative of each nucleotide in each of a j^{th} group of nucleotide positions i, for i equal 1 through k and for j equal 1 through m;
(b) for every group of nucleotide positions from j equal 2 through m, and every position from i equal 1 through k, adjusting the values ^{j}vᵢ₁, ^{j}vᵢ₂, ^{j}vᵢ₃, and ^{j}vᵢ₄ by repeatedly subtracting from each a first predetermined fraction of ^{j-1}vᵢ₁, ^{j-1}vᵢ₂, ^{j-1}vᵢ₃, and ^{j-1}vᵢ₄, respectively, until the ratio of the highest value in the set of ^{j}vᵢ₁ through ^{j}vᵢ₄, to the next highest value in the same set is greater than or equal to a predetermined factor, or until the repeatedly subtracted fractions have a sum equal to a second predetermined fraction; and
(c) generating a base call for position i in the j^{th} group based on results of the adjusting in (b).

29. The processor-readable medium of claim 28, wherein said base call generating instructions (c) comprises instructions for
assigning a base code corresponding to the highest value to position i in the j^{th} group whenever the highest value is greater than or equal to a predetermined minimum value and the ratio of the highest value in the set of ^{j}vᵢ₁ through ^{j}vᵢ₄, to the next highest value in the same set is greater than or equal to the predetermined factor, and
assigning a two-base ambiguity code corresponding to the highest value and the next highest value whenever the ratio is less than the predetermined factor and the highest value and the next highest value are each greater than or equal to the predetermined minimum value.

30. The processor-readable medium of claim 29, further comprising instructions for rejecting the signature whenever the number of ambiguity codes assigned is greater than one.

31. The processor-readable medium of claim 29, wherein said obtaining instructions (a) comprises instructions for adjusting values ^{j}vᵢ₁, ^{j}vᵢ₂, ^{j}vᵢ₃, and ^{j}vᵢ₄, for i equal 1 through k and for j equal 1 through m, for background noise.

32. The processor-readable medium of claim 31, wherein the background noise is computed as the average of the lowest three of ^{j}vᵢ₁, ^{j}vᵢ₂, ^{j}vᵢ₃, and ^{j}vᵢ₄, and wherein the computed background noise is subtracted from each of ^{j}vᵢ₁, ^{j}vᵢ₂, ^{j}vᵢ₃, and ^{j}vᵢ₄.

33. The processor-readable medium of claim 29, wherein the groups of positions, j=1 through m, are contiguous.

34. The processor-readable medium of claim 29, wherein m=3, 4 or 5.

35. The processor-readable medium of claim 34, wherein m=4.

36. The processor-readable medium of claim 29, wherein k=1, 2, 3, 4 or 5.

37. The processor-readable medium of claim 36, wherein k=2, 3 or 4.

38. The processor-readable medium of claim 37, wherein k=4.

39. The processor-readable medium of claim 29, wherein the predetermined factor is between about 2 and about 5.

40. The processor-readable medium of claim 31, wherein the predetermined minimum value is greater than 125% of the background noise.

41. The processor-readable medium of claim 29, wherein the first predetermined fraction is 1/50.

42. The processor-readable medium of claim 31, wherein the second predetermined fraction is set such that the highest value does not fall below 125% of the background noise.

43. A graphical user interface presented on a computer for facilitating interaction between a user and a computer-implemented method of determining a signature of a nucleotide sequence, the graphical user interface comprising:
(a) a data display area for displaying one or more displays of data; and
(b) a control area for displaying one or more selectable functions including
(i) a first function which when selected causes a graphical representation of the plurality of digital signal values to be displayed in the data display area, and
(ii) a second function which when selected causes a graphical representation of a plurality of sequence-containing microbeads to be displayed in the data display area.

44. The graphical user interface of claim 43, wherein the one or more selectable functions are represented by graphical push buttons displayed in the control area of the graphical user interface.

45. A graphical user interface presented on a computer for facilitating interaction between a user and a computer-implemented method of determining a signature of a nucleotide sequence, the graphical user interface comprising:
(a) an animation mode including a first main window having (i) a display area for displaying an animated image of a sequence-containing bead array, and a first control panel for displaying one or more selectable functions associated with the animation mode;
(b) an alignment mode including a second main window for aligning shifted images to show bead movement based on a comparison with a reference image, and a second control panel for displaying one or more selectable functions associated with the alignment mode; and
(c) a bead mode including a third main window for displaying a sequence-containing bead array, and one or more selectable functions for performing one or more base calling functions.

46. A method of determining a nucleotide sequence of a polynucleotide from a series of optical measurements comprising a plurality of groups, each group containing one or more sets of four optical measurements wherein each optical measurement of a set corresponds to a different one of deoxyadenosine, deoxyguanosine, deoxycytidine, or deoxythymidine, the groups of optical measurements being produced by successively ligating to and cleaving from the end of a polynucleotide signal-generating adaptor having protruding strands, and each optical measurement having a value, and each set of optical measurements corresponding to a separate nucleotide position of the protruding strand of a signal-generating adaptor, the method comprising the steps of:
adjusting the value of the optical measurement of each set within a group by repeatedly subtracting therefrom a predetermined fraction of the value of the corresponding optical measurement of the corresponding set obtained in the previous ligation until the ratio of the highest value to the next highest value in the same set is greater than or equal to a first predetermined fraction, or until the sum of the repeatedly subtracted fractions is less than or equal to a predetermined factor; and
assigning a base code to each set based on the results of the adjusting.
